(19) United States
(12) Patent Application Publication
(10) Pub. No.: US 2010/0273722 A1
(43) Pub. Date: Oct. 28, 2010

(54) MODIFIED MINIATURE PROTEINS

(75) Inventors: Alanna Shrader Schepartz, Wilton, CT (US); Abby M. Hodges, Upland, CA (US); Crystal Zellofrow, Apollo, PA (US); Joshua A. Kritzer, Groton, MA (US)

Correspondence Address: WOLF GREENFIELD & SACKS, P.C., 600 ATLANTIC AVENUE, BOSTON, MA, 02210-2206, US

(73) Assignee: Yale University, New Haven, CT (US)

(21) Appl. No.: 12/671,385

(22) PCT Filed: Apr. 16, 2008
(86) PCT No.: PCT/US08/04942

§ 371 Date: Jul. 2, 2010

Related U.S. Application Data
(60) Provisional application No. 60/963,744, filed on Aug. 6, 2007.

(51) Int. Cl. A61K 38/16; C07K 14/435

(52) U.S. Cl. 514/21.3; 530/324

(57) ABSTRACT

The present invention generally relates to modified miniature proteins, including modified avian pancreatic polypeptides (aPP) and modified pancreatic peptide YYs (PYY). One aspect of the invention is generally directed to various aPPs that have been modified such that they do not substantially form multimers in solution, for example through the addition of a proline switch. Another aspect of the invention is generally directed to modified PYYs, such as YY3. Yet another aspect of the invention is generally directed to composites of modified miniature proteins formed from portions of different miniature proteins such as aPP and/or PYY, optionally with a proline switch. Still other aspects of the invention are generally directed to methods of making such proteins, methods of using such proteins, kits involving such proteins, and the like.

### RELATED APPLICATIONS

This application claims the benefit of U.S. Provisional Patent Application Ser. No. 60/963,744, filed Aug. 6, 2007, entitled “Engineering a Monomeric Miniature Protein,” by Hodges, et al., incorporated herein by reference.

### GOVERNMENT FUNDING

Research leading to various aspects of the present invention were sponsored, at least in part, by the National Institutes of Health, Grant Nos. GM 65453 and 59843. The U.S. Government has certain rights in the invention.

### FIELD OF INVENTION

The present invention generally relates to modified miniature proteins, including modified avian pancreatic polypeptides and modified pancreatic peptide YYs.

### BACKGROUND

Many proteins recognize nucleic acids, other proteins or macromolecular assemblies using a partially exposed alpha helix. Within the context of a native protein fold, such alpha helices are usually stabilized by extensive tertiary interactions with residues that may be distant in primary sequence from both the alpha helix and from each other. With some exceptions, removal of these tertiary interactions destabilizes the alpha helix and results in molecules that neither fold nor function in macromolecular recognition. The ability to recapitulate or perhaps even improve on the recognition properties of an alpha helix within the context of a small molecule should find utility in the design of synthetic mimetics or inhibitors of protein function, or new tools for proteomics research.

Two fundamentally different approaches have been taken to bestow alpha helical structure on otherwise unstructured peptide sequences. One approach makes use of modified amino acids or surrogates that favor helix initiation or helix propagation. Some success has been realized by joining the i and i+7 positions of a peptide with a long-range disulfide bond to generate molecules whose helical structure was retained at higher temperatures. A second approach is to pare the extensive tertiary structure surrounding a given recognition sequence to generate the smallest possible molecule possessing function. This strategy has generated minimized versions of the Z domain of protein A (fifty-nine amino acids) and atrial natriuretic peptide (twenty-eight amino acids). The two minimized proteins, at thirty-three and fifteen amino acids, respectively, displayed relatively high biological activity. Despite this success, it is difficult to envision a simple and general application of this truncation strategy in the large number of cases where the alpha helical epitope is stabilized by residues scattered throughout the primary sequence.

In light of this limitation, a more flexible approach to protein minimization called protein grafting has been employed. Schematically, protein grafting involves removing residues required for molecular recognition from their native alpha helical context and grafting them on the scaffold provided by small yet stable proteins. Numerous researchers have engineered protein scaffolds to present binding residues on a relatively small peptide carrier. These scaffolds are small polypeptides onto which residues critical for binding to a selected target can be grafted. The grafted residues are arranged in particular positions such that the spatial arrangement of these residues mimics that which is found in the native protein. These scaffolding systems are commonly referred to as miniature proteins or miniproteins. A common feature is that the binding residues are known before the miniprotein is constructed.

Examples of these miniproteins include the thirty-seven amino acid protein charybdotoxin and the thirty-six amino acid protein, avian pancreatic peptide. Avian pancreatic polypeptide (aPP) is a polypeptide in which residues fourteen through thirty-two form an alpha helix stabilized by hydrophobic contacts with an N-terminal type II polyproline (PPII) helix formed by residues one through eight. Because of its small size and stability, aPP is a useful scaffold for protein grafting of alpha helical recognition epitopes.

### SUMMARY OF THE INVENTION

The present invention generally relates to modified miniature proteins, including modified avian pancreatic polypeptides and modified pancreatic peptide YYs. The subject matter of the present invention involves, in some cases, interrelated products, alternative solutions to a particular problem, and/or a plurality of different uses of one or more systems and/or articles.

In one aspect, the invention is directed to a composition. The composition comprises, according to one set of embodiments, the sequence APPLPPRNRPGEDASPEELSRYYASLRHYLNLVTRQRY (SEQ ID NO: 18). The composition, according to another set of embodiments, includes a protein having the sequence GPSQPTX¹PGDDAX²PEDLIRFYDNLQQYLNVVTRHRY (SEQ ID NO: 32), where X¹ and X² are each any amino acid residue.

In yet another set of embodiments, the composition comprises a protein having a total length of no more than about 40 amino acids and a sequence X¹X²X³, where X¹ comprises a sequence J¹PJ²J³J⁴J⁵J⁶ (SEQ ID NO: 20) or J¹PJ²J³PJ⁴J⁵J⁶J⁷ (SEQ ID NO: 21), X² is a hinge region comprising proline, at least about 50% of X³ comprises at least 10 residues of an alpha helix of PYY, and each of J¹-J⁷ is independently an amino acid residue.

The composition, in yet another set of embodiments, includes an avian pancreatic polypeptide (aPP) modified by substitution of one to four amino acid residues. In some cases, the modified miniature protein, when in phosphate-buffered saline solution at a concentration of 10⁻⁶ M, does not substantially form a multimer. In still another set of embodiments, the avian pancreatic polypeptide (aPP) is modified by the insertion of one to four amino acid residues.

According to another set of embodiments, the composition includes an avian pancreatic polypeptide (aPP) modified by substitution of one to four amino acid residues. In certain instances, at least one of the substitutions is the substitution of position 14 with proline.

In still another set of embodiments, the composition includes an avian pancreatic polypeptide (aPP) modified by substitution of one to four amino acid residues. In some cases, at least two of which substitutions are consecutive residues that are modified by switching their positions within the polypeptide.

Several method are disclosed herein of administering a subject with a compound for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

In another aspect, the present invention is directed to a method of making one or more of the embodiments described herein, for example, a modified avian pancreatic polypeptide or a modified pancreatic peptide YY. In another aspect, the present invention is directed to a method of using one or more of the embodiments described herein, for example, a modified avian pancreatic polypeptide or a modified pancreatic peptide YY.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1D illustrate various modified miniature proteins, in one embodiment of the invention;

FIGS. 2A and 3A illustrate isothermal CD analysis of certain miniature proteins, in various embodiments of the invention;

FIGS. 2B and 3B illustrate temperature-dependent CD analysis of certain miniature proteins, in another embodiment of the invention;

FIG. 4 illustrates sedimentation equilibrium analysis of certain miniature proteins, in yet another embodiment of the invention;

FIG. 5 illustrates temperature-dependent sedimentation equilibrium of a miniature protein, in still another embodiment of the invention;

FIGS. 6A-6C illustrate various modified miniature proteins, according to another embodiment of the invention;

FIGS. 7A-7C illustrate the binding of various miniature proteins, in yet another embodiment of the invention;

FIGS. 8A-8B illustrate Hck kinase activation by certain miniature proteins in vitro, in still another embodiment of the invention;

FIG. 8C illustrates Hck kinase activation by certain miniature proteins in live cells, in yet another embodiment of the invention;

FIGS. 9A-9B illustrate temperature-dependent CD analysis of certain miniature proteins, in another embodiment of the invention; and

FIGS. 10A-10C illustrate sedimentation equilibrium analysis of certain miniature proteins, in still another embodiment of the invention; and

FIG. 11 illustrates the binding of various miniature proteins to SH3-GSTcertain miniature proteins, in yet another embodiment of the invention.

### BRIEF DESCRIPTION OF THE SEQUENCES

SEQ ID NO: 1 is aPP, having the sequence GPSQPTYPGDDAPVEDLIRFYDNLQQYLNVVTRHRY;
SEQ ID NO: 2 is PYY, having the sequence YPAKPEAPGEDASPEELSRYYASLRHYLNLVTRQRY;

SEQ ID NO: 3 is pGolemi, having the sequence PFPPTPPGEEAPVEDLIRFYNDLQQYLNVV;

SEQ ID NO: 4 is p007, having the sequence GGSRATMPGDDAPVEDLKRFRNTLAARRSRARKAARA;

SEQ ID NO: 5 is aPP^{Y7A}, having the sequence GPS QPTAPGDDAPVEDLIRFYDNLQQYLNVVTRHRY;

SEQ ID NO: 6 is aPP^{F20A}, having the sequence GPSQPTYPGDDAPVEDLIRAYDNLQQYLNVVTRHRY;

SEQ ID NO: 7 is aPP^{Y21A}, having the sequence GPSQPTYPGDDAPVEDLIRFADNLQQYLNVVTRHRY;

SEQ ID NO: 8 is aPP^{Y7AT6E}, having the sequence CPSQPEAPGDDAPVEDLIRFYDNLQQYLNVVTRHRY;

SEQ ID NO: 9 is aPP^{Y7AQ25R}, having the sequence GPSQPTAPGDDAPVEDLIRFYDNLRQYLNVVTRHRY;

SEQ ID NO: 10 is aPP^{Y7AP13SV14P}, having the sequence GPSQPTAPGDDASPEDLIRFYDNLQQYLNVVTRHRY;

SEQ ID NO: 11 is aPP^{Y7AP13VV14P}, having the sequence GPSQPTAPGDDAVPEDLIRFYDNLQQYLNVVTRHRY;

SEQ ID NO: 12 is PP1, having the sequence APPLPPRNPGDDAPVEDLIRFYDNLQQYLNVVTRHRY;

SEQ ID NO: 13 is PP2, having the sequence APPLPPRNRGDDAPVEDLIRFYDNLQQYLNVVTRHRY;

SEQ ID NO: 14 is PP3, having the sequence APPLPPRNRPRDAPVEDLIRFYDNLQQYLNVVTRHRY;

SEQ ID NO: 15 is YY1, having the sequence APPLPPRNPGEDASPEELSRYYASLRHYLNLVTRQRY;

SEQ ID NO: 16 is YY2, having the sequence APPLPPRNRGEDASPEELSRYYASLRHYLNLVTRQRY;

SEQ ID NO: 17 is Peptide 1 (or APP12), having the sequence APPLPPRNRPRL;

SEQ ID NO: 18 is YY3, having the sequence APPLPPRNRPGEDASPEELSRYYASLRHYLNLVTRQRY;

SEQ ID NO: 19 is the Hck substrate peptide, having the sequence AEEEIYGEFEAKKKKG;

SEQ ID NO: 20 is the sequence XPXXPXXX, where each X is independently an amino acid residue;

SEQ ID NO: 21 is the sequence XPXXPXXXX, where each X is independently an amino acid residue;

SEQ ID NO: 22 is the sequence PPXXPXXX, where each X is independently an amino acid residue;

SEQ ID NO: 23 is the sequence PPXXPXXXX, where each X is independently an amino acid residue;

SEQ ID NO: 24 is the sequence XPXPPXXX, where each X is independently an amino acid residue;

SEQ ID NO: 25 is the sequence XPXPPXXXX, where each X is independently an amino acid residue;

SEQ ID NO: 26 is the sequence PPXPPXXX, where each X is independently an amino acid residue;

SEQ ID NO: 27 is the sequence PPXPPXXXX, where each X is independently an amino acid residue;

SEQ ID NO: 28 is the sequence PPLPPRNR;

SEQ ID NO: 29 is the sequence PEELSRYYASLRHYLN;

SEQ ID NO: 30 is the sequence GEDASPEELSRYYASLRHYLNLVTRQRY;

SEQ ID NO: 31 is the sequence GDDAPVEDLIRFYDNLQQYLNVVTRHRY;

SEQ ID NO: 32 is the sequence GPSQPTXPGDDAXPEDLIRFYDNLQQYLNVVTRHRY, where each X is independently an amino acid residue;

SEQ ID NO: 33 is the sequence SRYYASLRHYLNLVTRQRY; and
SEQ ID NO: 34 is the sequence IRFYDNLQQYLNVVTRHRY.

### DETAILED DESCRIPTION

The present invention generally relates to modified miniature proteins, including modified avian pancreatic polypeptides (aPP) and modified pancreatic peptide YYs (PYY). One aspect of the invention is generally directed to various aPPs that have been modified such that they do not substantially form multimers in solution, for example, through the addition of a proline switch. Another aspect of the invention is generally directed to modified PYYs, such as YY3. Yet another aspect of the invention is generally directed to composites of modified miniature proteins formed from portions of different miniature proteins such as aPP and/or PYY, optionally with a proline switch. Still other aspects of the invention are generally directed to methods of making such proteins, methods of using such proteins, kits involving such proteins, and the like.

Various aspects of the invention are generally directed to various miniature proteins, such as aPP or PYY, that has been modified such that the miniature protein does not substantially form multimers in solution, for example through the addition of a proline switch to the structure of the miniature protein. As used herein, the terms “miniature protein” or “miniprotein” refer to a relatively small protein containing at least a protein scaffold and one or more additional domains or regions that help to stabilize its tertiary structure. In some cases, the miniature protein may have a length of no more than 40 or 45 residues. For instance, in various embodiments, the miniature protein may have a length of 30, 31, 32, 33, 34, 35, 36, 37, 38, 39, or 40 residues.

The term “protein,” as used herein, is given its ordinary definition in the art, e.g., any of a group of complex organic compounds which contain carbon, hydrogen, oxygen, nitrogen and usually sulfur, which are widely distributed in plants and animals. Twenty different amino acids are commonly found in proteins and each protein has a unique, genetically defined amino acid sequence which determines its specific shape and function. The term “protein” is generally used herein interchangeably with the terms “peptide” and “polypeptide.” The term “protein scaffold” refers to a region or domain of a relatively small protein, such as a miniature protein, that has a conserved tertiary structural motif which can be modified to display one or more specific amino acid residues in a fixed conformation.

Non-limiting examples of miniature proteins include the PP fold protein scaffolds, which generally contain thirty-six amino acids and are the smallest known globular proteins. Despite their small size, PP fold proteins are stable and remain folded under physiological conditions. Some PP fold protein scaffolds of the invention comprise two anti-parallel helices, an N-terminal type II polyproline helix (PPII) between amino acid residues two and eight and an alpha helix between residues 14 and 31 and/or 32. The stability of the PP fold protein scaffolds of the invention derives predominantly from interactions between hydrophobic residues on the interior face of the alpha helix at positions 17, 20, 24, 27, 28, 30, and 31 and the residues on the two edges of the polyproline helix at positions 2, 4, 5, 7, and 8. In general, the residues responsible for stabilizing tertiary structure are not substituted in order to maintain the tertiary structure of the miniature protein or are compensated for using phage display.

Positions for grafting these binding site residues on the protein scaffold include, but are not limited to, positions on the solvent-exposed alpha-helical face of aPP. Substitutions of binding site residues may be made, in some cases, for residues involved in stabilizing the tertiary structure of the miniature protein. As used herein, the term “exposed on the alpha helix domain” means that an amino acid substituted, for example, into aPP or PYY, is available for association or interaction with another molecule and is not otherwise bound to or associated with another amino acid residue on the aPP or PYY. This term is used interchangeably with the term “solvent-exposed alpha helical face.”

Members of the PP fold family of protein scaffolds which are contemplated for use in the present invention include, but are not limited to, avian pancreatic polypeptide (aPP), Neuropeptide Y, lower intestinal hormone polypeptide and pancreatic peptide YY (PYY) (e.g., SEQ ID NO: 2). In one embodiment, the protein scaffold comprises the PP fold protein, avian pancreatic polypeptide (SEQ ID NO: 1). aPP is a PP fold polypeptide characterized by a short (eight residue) amino-terminal type II polyproline helix linked through a type I beta turn to an eighteen residue alpha helix. Because of its small size and stability, aPP is an excellent protein scaffold for, e.g., protein grafting of alpha-helical recognition epitopes.

One aspect of the invention is generally directed to modified PYYs, such as YY3 (SEQ ID NO: 18). YY3 is a PYY modified to include a “proline switch” region, which is a region located between the N-terminal type II polyproline helix (PPII) and the C-terminal alpha helix portions of the miniature protein that allows the protein to properly fold. Typically, the proline switch region is in a relatively unstructured portion of the molecule between the two helical structures, and contains one or more proline residues. In some cases, the proline switch region may be as small as a single residue. However, it should be understood that YY3 is presented here by way of example only, and that the invention includes virtually any miniature protein modified to include a proline switch region between the N-terminal type II polyproline helix and the C-terminal alpha helix portions of the miniature protein. For instance, in one set of embodiments, the miniature protein may contain a first region containing an N-terminal type II polyproline helix, a second region containing a proline switch region, and a third region containing an alpha helix. As another example, in some cases, a molecule may include a PYY sequence solely after the binding epitope ends at residue 10.

The N-terminal type II polyproline helix is typically 7, 8, 9, 10, 11, or 12 residues long, and may contain, in some embodiments, 1, 2, 3, 4, 5, or more proline residues. For instance, in one embodiment, the N-terminal type II polyproline helix contains a sequence XPXPXXX (SEQ ID NO: 20) or XPXPXXXX (SEQ ID NO: 21), where P is proline and each X is independently an amino acid residue (which may be P, or another amino acid residue). For example, the N-terminal type II polyproline helix may have sequences such as PPXXPXXX (SEQ ID NO: 22), PPXXPXXXX (SEQ ID NO: 23), XPXPPXXX (SEQ ID NO: 24), XPXPPXXXX (SEQ ID NO: 25), PPXPPXXX (SEQ ID NO: 26), PPXPPXXXX (SEQ ID NO: 27), etc. A specific example of an N-terminal type II polyproline helix sequence is PPLPPRNR (SEQ ID NO: 28).

The C-terminal portion of the molecule may comprise an alpha helix, and the alpha helix may be any length within the C-terminal portion of the molecule. For instance, the alpha helix may have a length of at least 10 residues, at least 15 residues, or at least 20 residues within the C-terminal portion. As other examples, at least about 40%, at least about 50%, at least about 60%, at least about 70%, at least about 80%, or at least about 90% of the C-terminal portion of the molecule may have an alpha helical configuration.

In some cases, the C-terminal portion of the miniature protein may be based on the alpha helix portion of aPP or PYY (e.g., SEQ ID NO: 31 or SEQ ID NO: 30). In other cases, however, the C-terminal portion may be modified from one of these in some way. For example, 1, 2, 3, 4, 5, 6, or more of the residues in the original aPP or PYY portions may be substituted with an arginine residue. In some cases, the residues that are substituted with arginine residues may be ones located on the outer portion of the protein when the protein is properly folded. Examples of such residues include those located in positions 19, 20, 24, 26, 27, 30, or 31 of the molecule.

Other substitutions are also contemplated, for example substitutions within the molecule that contribute to aPP/PYY folding. For instance, positions such as those positions indicated by square brackets in FIG. 6B may be present on the outer portion of the protein when the protein is folded correctly, and one or more of these may be substituted in some fashion.

In another aspect, the present invention contemplates a miniature protein, such as an avian pancreatic polypeptide, modified by the substitution of one to four amino acid residues, in such a way that the miniature protein is monomeric, e.g., such that the modified miniature protein, when in Tris-buffered saline solution or phosphate-buffered saline solution at a concentration of 10⁻⁶ M, does not substantially form a multimer. As used herein, a “multimer” and a “monomer” are given their ordinary meanings as used in the art, e.g., an aggregate of multiple copies of a miniature protein formed in solution. Thus, for instance, a dimer is two copies of a miniature protein that have become associated together when in solution, e.g., when in Tris-buffered saline solution at a concentration of 10⁻⁶ M; other order multimers (3, 4, 5, 6, etc.) are also possible in some cases. A monomer, of course, is a protein that forms only low levels of such aggregates when in such solutions (for instance, below about 10⁻⁶ M in solution). A monomeric protein in solution can be detected using any technique known to those of ordinary skill in the art, for example, using sedimentation equilibrium analysis and curve fitting to a best-fit monomer-n-mer model, as is discussed in Example 1. In one set of embodiments, at least one of the substituted amino acid residues is a proline. For instance, in some cases, a miniature protein may be modified to include a proline, which may define a “proline switch region” located between the N-terminal type II polyproline helix (PPII) and the C-terminal alpha helix portions of the miniature protein. The proline switch region, when added, may allow the miniature protein to properly fold. Typically the proline switch region includes positions 13, 14, and/or 15. In some cases, the proline switch allows the molecule to refold into a polyproline fold once the dimerization has been removed, i.e., the proline switch may help to stabilize folding in the molecule. It should also be noted that the addition of a proline switch region may increase the length of the molecule.

In addition, other modifications to the miniature protein are also possible, e.g., substitutions of positions 7 and/or 20 on an aPP to further limit dimerization. Other possible substitutions include, but are not limited to, positions 6 or 25, etc. In some cases, the miniature protein may be prepared by switching a proline from a nearby (typically consecutive) position within the molecule, e.g., from position 15 to 14, from position 12 to 13, etc.

Examples of miniature proteins modified to include a proline switch region include, but are not limited to, aPP^{Y7A} (SEQ ID NO: 5), aPP^{F20A} (SEQ ID NO: 6), aPP^{Y21A} (SEQ ID NO: 7), aPP^{Y7AAT6E} (SEQ ID NO: 8), aPP^{Y7AQ25R} (SEQ ID NO: 9), aPP^{Y7AP13SV14P} (SEQ ID NO: 10), or aPP^{Y7AP13W14P} (SEQ ID NO: 11), as are discussed in detail below. As another example, the miniature protein may include a sequence such as GPSQPTXPGDDAXPEDLIRFYDNLQQYLNVVTRHRY (SEQ ID NO: 32), where each X can independently be any amino acid residue, e.g., tyrosine, valine, serine, etc. In some cases, the first X may be a nonaromatic amino acid. In certain instances, the second X may be an amino acid capable of hydrogen bond donation.

Successfully modified miniature proteins can be readily determined (qualitatively and/or quantitatively) by the ability to remain as monomers in solution (which can be determined as discussed above, e.g., using sedimentation equilibrium analysis, and/or by determining their activity or conformation, for instance, using wavelength-dependent circular dichroism (CD) spectra, as discussed in the examples).

In some aspects, the modified miniature proteins are able to associate with (or bind to) specific sequences of DNA or other proteins. These miniature proteins may be able to bind, for example, to DNA or other proteins with high affinity and selectivity. As used herein, the term “bind” or “binding” refers to the specific association or other specific interaction between two molecular species, such as, but not limited to, protein-DNA interactions and protein-protein interactions, for example, the specific association between proteins and their DNA targets, receptors and their ligands, enzymes and their substrates, etc. It is contemplated that such association may be mediated through specific sites on each of two (or more) interacting molecular species. Binding can be mediated by structural and/or energetic components. In some cases, the latter will comprise the interaction of molecules with opposite charges.

In one set of embodiments, the invention involves a technique known as protein grafting. Protein grafting has generally been described in U.S. patent application Ser. No. 09/840,085, filed Apr. 24, 2001, entitled “Modified Avian Pancreatic Polypeptide Miniature Binding Proteins,” by A. S. Shrader, et al., now U.S. Pat. No. 7,297,762, issued Nov. 20, 2007, incorporated herein by reference. Briefly, protein grafting identifies binding site residues from a globular protein that is able to participate in binding-type associations between that protein and its specific binding partners, then the residues are grafted onto a small but stable protein scaffold. As used herein, the term “binding site” refers to the reactive region or domain of a molecule that directly participate in its specific binding with another molecule. For example, when referring to the binding site on a protein or nucleic acid, binding occurs as a result of the presence of specific amino acids or nucleotide sequence, respectively, that interact with the other molecule. Examples of protein scaffolds of the invention comprise members of the pancreatic fold (PP fold) protein family, particularly avian pancreatic polypeptide (aPP) or pancreatic peptide YY (PYY).

Thus, in one aspect, a modified miniature protein may be able to associate with or bind to a specific sequence of DNA. In some embodiments, the DNA sequence may comprise sites for known proteins that bind to that specific DNA sequence (contemplated known proteins would be, e.g., a promotor or regulator). For example, in the design of a DNA-binding miniature protein, the amino acid residues of a known protein that participate in binding or other association of the protein to that particular DNA sequence are identified.

In some embodiments of the present invention, the relevant binding residues are identified using three-dimensional models of a protein or protein complex based on crystallographic studies while in other embodiments they are identified by studies of deletion or substitution mutants of the protein. The residues that participate in binding of the protein to the specific DNA sequence are then grafted onto those positions of the miniature protein that are not necessary to maintain the tertiary structure of the protein scaffold to form the DNA-binding miniature protein. The identification of such positions can readily be determined empirically by persons of ordinary skill in the art. Other embodiments of the present invention involve the screening of a library of modified miniproteins that contain peptide species capable of specific association or binding to that specific DNA (or, in other cases, protein) sequence or motif.

Generally, it is contemplated that any potential binding site on a DNA sequence can be targeted using the DNA binding miniature proteins of the invention. Certain embodiments include miniature proteins having helical structures which bind to a DNA binding site. In some embodiments, the binding involves a basic region leucine zipper (bZIP) structure, while in other embodiments the structure involves a basic-helix-loop-helix (bHLH) structure. In another embodiment, the binding involves a structure like those found in homeodomain proteins. Example bZIP structures include, but are not limited to, those found in GCN4 and C/EBP-delta, and example bHLH structures include, but are not limited to, those found in Max, Myc and MyoD. Example homeodomain structures include, but are not limited to, those found in the Q50 engrailed variant protein.

In some aspects, a miniature protein is produced and selected using a phage display method. In such a method, display of recombinant miniature proteins on the surface of viruses which infect bacteria (bacteriophage or phage) make it possible to produce soluble, recombinant miniature proteins having a wide range of affinities and kinetic characteristics. To display the miniature proteins on the surface of phage, a synthetic gene encoding the miniature protein is inserted into the gene encoding a phage surface protein (pIII) and the recombinant fusion protein is expressed on the phage surface. Variability may be introduced into the phage display library to select for miniature proteins which not only maintain their tertiary, helical structure but which also display increased affinity for a preselected target because the critical (or contributing but not critical) binding residues are optimally positioned on the helical structure.

Since the recombinant proteins on the surface of the phage are functional, phage bearing miniature proteins that bind with high-affinity to a particular target DNA or protein can be separated from non-binding or lower affinity phage by using techniques such as antigen affinity chromatography. Mixtures of phage are allowed to bind to the affinity matrix, non-binding or lower affinity phage are removed by washing, and bound phage are eluted by treatment with acid or alkali. Depending on the affinity of the miniature protein for its target, enrichment factors of twenty-fold to a million-fold are obtained by a single round of affinity selection. By infecting bacteria with the eluted phage, however, more phage can be grown and subjected to another round of selection. In this way, an enrichment of a thousand-fold in one round becomes a million-fold in two rounds of selection. Thus, even when enrichments in each round are low, multiple rounds of affinity selection leads to the isolation of rare phage and the genetic material contained within which encodes the sequence of the domain or motif of the recombinant miniature protein that binds or otherwise specifically associates with it binding target.

Accordingly, in various embodiments of the invention, the methods disclosed herein are used to produce a phage expression library encoding miniature proteins capable of binding to a DNA or to a protein that has already been selected using the protein grafting procedure described above. In such embodiments, phage display can be used to identify miniature proteins that display an even higher affinity for a particular target DNA or protein than that of the miniature proteins produced without the aid of phage display. In yet another embodiment, the invention encompasses a universal phage display library that can be designed to display a combinatorial set of epitopes or binding sequences to permit the recognition of nucleic acids, proteins or small molecules by a miniature protein without prior knowledge of the natural epitope or specific binding residues or motifs natively used for recognition and association.

Various structural modifications also are contemplated for the present invention that, for example, include the addition of restriction enzyme recognition sites into the polynucleotide sequence encoding the miniature protein that enable genetic manipulation of these gene sequences. Accordingly, the re-engineered miniature proteins can be ligated, for example, into an M13-derived bacteriophage cloning vector that permits expression of a fusion protein on the phage surface. These methods allow for selecting phage clones encoding fusion proteins that bind a target ligand and can be completed in a rapid manner allowing for high-throughput screening of miniature proteins to identify the miniature protein with the highest affinity and selectivity for a particular target.

According to the methods of the invention, a library of phage displaying modified miniature proteins is incubated with the immobilized target DNA or proteins to select phage clones encoding miniature proteins that specifically bind to or otherwise specifically associate with the immobilized DNA or protein. This procedure involves immobilizing a oligonucleotide or polypeptide sample on a solid substrate. The bound phage are then dissociated from the immobilized oligonucleotide or polypeptide and amplified by growth in bacterial host cells. Individual viral plaques, each expressing a different recombinant miniature protein, are expanded to produce amounts of protein sufficient to perform a binding assay. The DNA encoding this recombinant binding protein can be subsequently modified for ligation into a eukaryotic protein expression vector. The modified miniature protein, adapted for expression in eukaryotic cells, is ligated into a eukaryotic protein expression vector.

In another aspect, the invention encompasses miniature proteins that bind to other proteins and methods for making these miniature proteins. The binding of the miniature proteins modulates protein-protein and/or protein-ligand interactions. Thus, in some embodiments the binding blocks the association (or specific binding) of ligands and receptors. The ligand can be either another protein but also can be any other type of molecule such as a chemical substrate. In one embodiment of the present invention, making the protein-binding miniature protein of the invention involves determining the amino acid residues which are essential to binding of the ligand protein to its target receptor protein. In some embodiments, these essential residues are identified using three-dimensional models of a protein or protein complex which binds to or interacts with another protein based on crystallographic studies while in other embodiments they are identified by studies of deletion or substitution mutants of the protein. The residues that participate in binding of the protein to are then grafted onto those positions which are not necessary to maintain the tertiary structure of the protein scaffold to form the protein-binding miniature protein.

The miniature proteins of the present invention further include conservative variants of the miniature proteins herein described, according to another aspect. As used herein, a “conservative variant” refers to alterations in the amino acid sequence that do not substantially and adversely affect the binding or association capacity of the protein. A substitution, insertion or deletion is said to adversely affect the miniature protein when the altered sequence prevents or disrupts a function or activity associated with the protein. For example, the overall charge, structure or hydrophobic-hydrophilic properties of the miniature protein can be altered without adversely affecting an activity. Accordingly, the amino acid sequence can be altered, for example to render the peptide more hydrophobic or hydrophilic, without adversely affecting the activities of the miniature protein.

These variants, though possessing a slightly different amino acid sequence than those recited above, will still have the same or similar properties associated with any of the miniature proteins discussed herein, for instance, SEQ ID NOs: 1, 2, 3, 4, 5, 6, 7, 9, 10, 11, 12, 13, 14, 15, 16, 17, or 18. Ordinarily, the conservative substitution variants, will have an amino acid sequence having at least 75%, at least 80%, at least 85%, at least 90%, at least 95% amino acid, at least 98%, or at least 99% sequence identity with any of the miniature proteins discussed herein, for example, SEQ ID NOs: 1, 2, 3, 4, 5, 6, 7, 9, 10, 11, 12, 13, 14, 15, 16, 17, or 18. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N-terminal, C-terminal or internal extensions, deletions, or insertions into the peptide sequence shall not be construed as affecting homology.

Thus, the miniature proteins of the present invention include molecules comprising any of the amino acid sequences discussed herein, including SEQ ID NOs: 1, 2, 3, 4, 5, 6, 7, 9, 10, 11, 12, 13, 14, 15, 16, 17, or 18; fragments thereof having a consecutive sequence of at least about 20, 25, 30, 35 or more amino acid residues of the miniature proteins of the invention; amino acid sequence variants of such sequences wherein at least one amino acid residue has been inserted N- or C-terminal to, or within, the disclosed sequence; amino acid sequence variants of the disclosed sequences, or their fragments as defined above, that have been substituted by another residue. Contemplated variants further include those derivatives wherein the protein has been covalently modified by substitution, chemical, enzymatic, or other appropriate means with a moiety other than a naturally occurring amino acid (for example, a detectable moiety such as an enzyme or radioisotope).

The present invention further provides, in another aspect, nucleic acid molecules that encode any of the amino acid sequences discussed herein, including SEQ ID NOs: 1, 2, 3, 4, 5, 6, 7, 9, 10, 11, 12, 13, 14, 15, 16, 17, or 18 and the related miniature proteins herein described, preferably in isolated form. As used herein, “nucleic acid” includes cDNA and mRNA, as well as nucleic acids based on alternative backbones or including alternative bases whether derived from natural sources or synthesized. Those of ordinary skill in the art, given an amino acid sequence, will be able to generate corresponding nucleic acid sequences that can be used to generate the amino acid sequence, using no more than routine skill. As used herein, a nucleic acid molecule is said to be “isolated” when the nucleic acid molecule is substantially separated from contaminant nucleic acid encoding other polypeptides from the source of nucleic acid.

The present invention also provides fragments of the encoding nucleic acid molecule. As used herein, a “fragment of an encoding nucleic acid molecule” refers to a portion of the entire protein encoding sequence of the miniature protein. The size of the fragment will be determined by the intended use. For example, if the fragment is chosen so as to encode an active portion of the protein, the fragment will need to be large enough to encode the functional region(s) of the protein. The appropriate size and extent of such fragments can be determined empirically by persons skilled in the art.

Modifications to the primary structure itself by deletion, addition, or alteration of the amino acids incorporated into the protein sequence during translation can be made without destroying the activity of the miniature protein. Such substitutions or other alterations result in miniature proteins having an amino acid sequence encoded by a nucleic acid falling within the contemplated scope of the present invention.

The present invention further provides, in some embodiments, recombinant DNA molecules that contain a coding sequence. As used herein, a “recombinant DNA molecule” is a DNA molecule that has been subjected to molecular manipulation. Methods for generating recombinant DNA molecules are well known in the art, for example, see Sambrook et al., (1989) Molecular Cloning: A Laboratory Manual, Cold Spring Harbor Laboratory Press. In some recombinant DNA molecules, a coding DNA sequence is operably linked to expression control sequences and vector sequences.

The choice of vector and expression control sequences to which one of the protein family encoding sequences of the present invention is operably linked depends directly, as is well known in the art, on the functional properties desired (e.g., protein expression, and the host cell to be transformed). A vector of the present invention may be at least capable of directing the replication or insertion into the host chromosome, and preferably also expression, of the structural gene included in the recombinant DNA molecule.

Expression control elements that are used for regulating the expression of an operably linked miniature protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extra-chromosomal in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical of bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Any suitable prokaryotic host can be used to express a recombinant DNA molecule encoding a protein of the invention.

Expression vectors compatible with eukaryotic cells, preferably those compatible with vertebrate cells, can also be used to form recombinant DNA molecules that contains a coding sequence. Eukaryotic cell expression vectors are well known in the art and are available from several commercial sources. Typically, such vectors are provided containing convenient restriction sites for insertion of the desired DNA segment.

Eukaryotic cell expression vectors used to construct the recombinant DNA molecules of the present invention may further include a selectable marker that is effective in an eukaryotic cell, preferably a drug resistance selection marker. An example drug resistance marker is the gene whose expression results in neomycin resistance, i.e., the neomycin phosphotransferase (neo) gene. Alternatively, the selectable marker can be present on a separate plasmid, the two vectors introduced by co-transfection of the host cell, and transfectants selected by culturing in the appropriate drug for the selectable marker.

The present invention further provides, in yet another aspect, host cells transformed with a nucleic acid molecule that encodes a miniature protein of the present invention. The host cell can be either prokaryotic or eukaryotic. Eukaryotic cells useful for expression of a miniature protein of the invention are not limited, so long as the cell line is compatible with cell culture methods and compatible with the propagation of the expression vector and expression of the gene product.

Transformation of appropriate cell hosts with a recombinant DNA molecule encoding a miniature protein of the present invention is accomplished by well known methods that typically depend on the type of vector used and host system employed. With regard to transformation of prokaryotic host cells, electroporation and salt treatment methods can be employed (see, for example, Sambrook et al., (1989) Molecular Cloning: A Laboratory Manual, Cold Spring Harbor Laboratory Press). With regard to transformation of vertebrate cells with vectors containing recombinant DNA, electroporation, cationic lipid or salt treatment methods can be employed (see, for example, Graham et al., (1973) Virology 52, 456-467; Wigler et al., (1979) Proc. Natl. Acad. Sci. USA 76, 1373-1376).

Successfully transformed cells (cells that contain a recombinant DNA molecule of the present invention), can be identified by well known techniques including the selection for a selectable marker. For example, cells resulting from the introduction of a recombinant DNA of the present invention can be cloned to produce single colonies. Cells from those colonies can be harvested, lysed and their DNA content examined for the presence of the recombinant DNA using a method such as that described by Southern, (1975) J. Mol. Biol. 98, 503-517 or the proteins produced from the cell assayed via an immunological method.

The present invention further provides, in still another aspect, methods for producing a miniature protein of the invention using nucleic acid molecules herein described. In general terms, the production of a recombinant form of a protein typically involves the following steps: a nucleic acid molecule is obtained that encodes a protein of the invention, such as the nucleic acid molecule encoding any of the miniature proteins described herein, including SEQ ID NOs: 1, 2, 3, 4, 5, 6, 7, 9, 10, 11, 12, 13, 14, 15, 16, 17, or 18. The nucleic acid molecule may then be placed in operable linkage with suitable control sequences, as described above, to form an expression unit containing the protein open reading frame. The expression unit is used to transform a suitable host and the transformed host is cultured under conditions that allow the production of the recombinant miniature protein. Optionally the recombinant miniature protein is isolated from the medium or from the cells; recovery and purification of the protein may not be necessary in some instances where some impurities may be tolerated.

Each of the foregoing steps can be done in a variety of ways. The construction of expression vectors that are operable in a variety of hosts is accomplished using appropriate replicons and control sequences, as set forth above. The control sequences, expression vectors, and transformation methods are dependent on the type of host cell used to express the gene. Suitable restriction sites, if not normally available, can be added to the ends of the coding sequence so as to provide an excisable gene to insert into these vectors. A artisan of ordinary skill in the art can readily adapt any host/expression system known in the art for use with the nucleic acid molecules of the invention to produce a recombinant miniature protein.

In another aspect, the present invention provides methods for use in isolating and identifying binding partners of the miniature proteins of the invention. In some embodiments, a miniature protein of the invention is mixed with a potential binding partner or an extract or fraction of a cell under conditions that allow the association of potential binding partners with the protein of the invention. After mixing, peptides, polypeptides, proteins or other molecules that have become associated with a miniature protein of the invention are separated from the mixture. The binding partner bound to the protein of the invention can then be removed and further analyzed. To identify and isolate a binding partner, the entire miniature protein can be used. Alternatively, a fragment of the miniature protein which contains the binding domain can be used.

As used herein, a “cellular extract” refers to a preparation or fraction which is made from a lysed or disrupted cell. A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the a miniature protein of the invention under conditions in which association of the miniature protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density-sediment centrifugation can be used.

After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the miniature protein of the invention can be immobilized on a solid support. For example, the miniature protein can be attached to a nitrocellulose matrix or acrylic beads. Attachment of the miniature protein to a solid support aids in separating peptide-binding partner pairs from other constituents found in the extract. The identified binding partners can be either a single DNA molecule or protein or a complex made up of two or more proteins. Alternatively, binding partners may be identified using the alkaline phosphatase fusion assay according to the procedures of Flanagan & Vanderhaeghen, (1998) Annu. Rev. Neurosci. 21, 309-345 or Takahashi et al., (1999) Cell 99, 59-69; the Far-Western assay according to the procedures of Takayama et al., (1997) Methods Mol. Biol. 69, 171-184, or Sauder et al., J. Gen. Virol. (1996) 77, 991-996 or identified through the use of epitope tagged proteins or GST fusion proteins.

In another embodiment, the nucleic acid molecules encoding a miniature protein of the invention can be used in a yeast two-hybrid system. The yeast two-hybrid system has been used to identify other protein partner pairs and can readily be adapted to employ the nucleic acid molecules herein described (see, e.g., Stratagene Hybrizap® two-hybrid system).

According to some aspects, the miniature proteins of the invention are useful for drug screening to identify agents capable of binding to the same binding site as the miniature proteins. The miniature proteins are also useful for diagnostic purposes to identify the presence and/or detect the levels of DNA or protein that binds to the miniature proteins of the invention. In one diagnostic embodiment, the miniature proteins of the invention are included in a kit used to detect the presence of a particular DNA or protein in a biological sample. The miniature proteins of the invention also have therapeutic uses in the treatment of disease associated with the presence of a particular DNA or protein. In one therapeutic embodiment, the miniature proteins can be used to bind to DNA to promote or inhibit transcription, while in another therapeutic embodiment, the miniature proteins bind to a protein resulting in inhibition or stimulation of the protein.

In another aspect, the miniature proteins may be used to modulate mammalian cell migration. Therefore, these miniature proteins can be important therapeutic compounds for diseases such as cancer cell metastasis, immune regulation, inflammatory disease, and neurodegenerative disorders. As used herein, the term “modulate” refers to an alteration in the association between two molecular species, for example, the effectiveness of a biological agent to interact with its target by altering the characteristics of the interaction in a competitive or non-competitive manner.

In some aspects of the invention, miniature proteins of the invention are administrated to a subject in an effective amount to inhibit (completely or partially) migration of a tumor cell across a barrier, thereby forming an eclipse. The invasion and metastasis of cancer is a complex process which involves changes in cell adhesion properties which allow a transformed cell to invade and migrate through the extracellular matrix (ECM) and acquire anchorage-independent growth properties. Some of these changes occur at focal adhesions, which are cell/ECM contact points containing membrane-associated, cytoskeletal, and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers. Thus the term “metastasis” as used herein refers to the invasion and migration of tumor cells away from the primary tumor site.

Miniature proteins of the invention are also useful for treating and/or preventing disorders associated with inflammation in a subject. For example, when an EnaNASP protein activity is induced in immune or hematopoetic cells, the ability of the cells to migrate is reduced. Thus, the subject minature proteins can induce activity of an EnaNASP protein in immune cells such that inflammatory disorders and ischemic diseases are prevented or treated.

Inflammatory disorders and ischemic diseases are characterized by inflammation associated with neutrophil migration to local tissue regions that have been damaged or have otherwise induced neutrophil migration and activation. While not intending to be bound by any particular theory, it is believed that excessive accumulation of neutrophils resulting from neutrophil migration to the site of injury, causes the release toxic factors that damage surrounding tissue. When the inflammatory disease is an acute stroke a tissue which is often damaged by neutrophil stimulation is the brain. As the active neutrophils accumulate in the brain an infarct develops.

An “inflammatory disease or condition” as used herein refers to any condition characterized by local inflammation at a site of injury or infection and includes autoimmune diseases, certain forms of infectious inflammatory states, undesirable neutrophil activity characteristic of organ transplants or other implants and virtually any other condition characterized by unwanted neutrophil accumulation at a local tissue site. These conditions include but are not limited to meningitis, cerebral edema, arthritis, nephritis, adult respiratory distress syndrome, pancreatitis, myositis, neuritis, connective tissue diseases, phlebitis, arteritis, vasculitis, allergy, anaphylaxis, ehrlichiosis, gout, organ transplants and/or ulcerative colitis.

An “ischemic disease or condition” as used herein refers to a condition characterized by local inflammation resulting from an interruption in the blood supply to a tissue due to a blockage or hemorrhage of the blood vessel responsible for supplying blood to the tissue such as is seen for myocardial or cerebral infarction. A cerebral ischemic attack or cerebral ischemia is a form of ischemic condition in which the blood supply to the brain is blocked. This interruption in the blood supply to the brain may result from a variety of causes, including an intrinsic blockage or occlusion of the blood vessel itself, a remotely originated source of occlusion, decreased perfusion pressure or increased blood viscosity resulting in inadequate cerebral blood flow, or a ruptured blood vessel in the subarachnoid space or intracerebral tissue.

It has been discovered that mammalian cell migration can be induced by depleting the cell of functional Ena/VASP protein. Therefore, miniature proteins of the invention can be useful for regeneration of tissue, including wound healing and neuroregeneration, or prevention or treatment of neurodegenerative disease.

A “wound” as used herein, means a trauma to any of the tissues of the body, especially that caused by physical means. The wound healing process involves a complex cascade of biochemical and cellular events to restore tissue integrity following an injury. The wound healing process is typically characterized by four stages: 1) hemostasis; 2) inflammation; 3) proliferation; and 4) remodeling. The miniature proteins of the invention can be useful for promoting wound healing by promoting cellular migration and thus remodeling. In one set of embodiments, the methods of the invention are useful for treating a wound to the dermis or epidermis, e.g., a burn or tissue transplant, injury to the skin. Further, the methods of the invention may be used in the process of wound healing as well as tissue generation. When the methods of the invention are used to promote wound healing, cells may be manipulated to alter Ena/VASP activity in vitro and then added to the site of the wound or alternatively the cells present at the site of the wound may be manipulated in vivo to alter the activity of the Ena/VASP proteins in order to promote cellular movement. When the methods are used to promote tissue generation, cells can be manipulated and grown in vitro on a scaffold and then implanted into the body or alternatively the scaffold may be implanted in the body, or it may be a naturally occurring scaffold and cells manipulated in vivo or in vitro can be used to generate the tissue.

Another aspect of the invention involves methods for tissue regeneration, which are particularly applicable to growth of neuronal cells. Thus, the invention contemplates the treatment of subjects having or at risk of developing neurodegenerative disease in order to cause neuroregeneration. Neuronal cells include both central nervous system (CNS) neurons and peripheral nervous system (PNS) neurons. There are many different neuronal cell types. Examples include, but are not limited to, sensory and sympathetic neurons, cholinergic neurons, dorsal root ganglion neurons, and proprioceptive neurons (in the trigeminal mesencephalic nucleus), ciliary ganglion neurons (in the parasympathetic nervous system). A person of ordinary skill in the art will be able to easily identify neuronal cells and distinguish them from non-neuronal cells such as glial cells, typically utilizing cell-morphological characteristics, expression of cell-specific markers, and secretion of certain molecules.

“Neurodegenerative disorder” is defined herein as a disorder in which progressive loss of neurons occurs either in the peripheral nervous system or in the central nervous system. Examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapie, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor). and Wemicke-Korsakoffs related dementia. Neurodegenerative diseases affecting sensory neurons include Friedreich's ataxia, diabetes, peripheral neuropathy, and retinal neuronal degeneration. Neurodegenerative diseases of limbic and cortical systems include cerebral amyloidosis, Pick's atrophy, and Retts syndrome. The foregoing examples are not meant to be comprehensive but serve merely as an illustration of the term “neurodegenerative disorder.”

Miniature proteins of the invention may be administrated to cells of a subject to treat or prevent diseases (e.g., cancer metastasis or inflammatory disorders) alone or in combination with the administration of other therapeutic compounds for the treatment or prevention of these disorders.

In certain aspects, miniature proteins of the invention are useful for diagnostic purposes to identify the presence and/or detect the levels of a target protein that binds to the miniature proteins of the invention. For example, miniature proteins of the invention can be used to detect the levels of an Ena/VASP protein due to its high affinity and high specifity. Miniature proteins of this method can be labeled with a detectable marker. A wide range of detectable markers can be used, including but not limited to biotin, a fluorogen, an enzyme, an epitope, a chromogen, or a radionuclide. The method for detecting the label will depend on the nature of the label and can be any known in the art, e.g., film to detect a radionuclide, an enzyme substrate that gives rise to a detectable signal to detect the presence of an enzyme, antibody to detect the presence of an epitope, etc.

In a specific diagnostic embodiment, miniature proteins of the invention are included in a kit used to detect the presence of a particular protein (e.g., an Ena/VASP protein) in a biological sample.

In certain aspects, therapeutic compounds of the present invention (e.g., miniature proteins) are formulated with a pharmaceutically acceptable carrier. Miniature proteins of the present invention can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the miniature proteins include those suitable for oral/nasal, topical, parenteral and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Methods of preparing these formulations or compositions include combining one compound and a carrier and, optionally, one or more accessory ingredients. In general, the formulations are prepared by combining a compound with a liquid carrier, or a finely divided solid carrier, or both, and then, if necessary, shaping the product.

Formulations of the miniature proteins suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound as an active ingredient. A compound may also be administered as a bolus, electuary or paste.

In solid dosage forms for oral administration (capsules, tablets, pills, dragees, powders, granules, and the like), a miniature protein is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and/or (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of a miniature protein include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds (e.g., miniature proteins), may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Methods of the invention can be administered topically in some embodiments, either to skin or to mucosal membranes (e.g., those on the cervix and vagina). This offers the greatest opportunity for direct delivery to tumor with the lowest chance of inducing side effects. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration of a compound (e.g., a miniature protein) include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a therapeutic compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Injectable depot forms are made by forming microencapsule matrices of the compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations of the compounds for intravaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Optionally, such formulations suitable for vaginal administration also include pessaries, tampons, creams, gels, pastes, foams or spray formulations containing such carriers as are known in the art to be appropriate.

The following documents are incorporated herein by reference: U.S. Provisional Patent Application Ser. No. 60/963,744, filed Aug. 6, 2007, entitled “Engineering a Monomeric Miniature Protein,” by Hodges, et al., incorporated herein by reference; U.S. patent application Ser. No. 09/840,085, filed Apr. 24, 2001, entitled “Modified Avian Pancreatic Polypeptide Miniature Binding Proteins,” by A. S. Shrader, et al., now U.S. Pat. No. 7,297,762, issued Nov. 20, 2007; U.S. patent application Ser. No. 11/009,101, filed Dec. 10, 2004, entitled “Protein Binding Miniature Proteins and Uses Thereof,” by D. Golemi-Kotra, et al., published as U.S. Patent Application Publication No. 2005/0287643 on Dec. 29, 2005; and U.S. patent application Ser. No. 10/982,727, filed Nov. 4, 2004, entitled “Protein Binding Miniature Proteins,” by A. S. Shrader, et al., published as U.S. Patent Application Publication No. 2005/0287542 on Dec. 29, 2005.

The following examples are intended to illustrate certain embodiments of the present invention, but do not exemplify the full scope of the invention.

### Example 1

The stability, size, and structure of avian pancreatic polypeptide (aPP) makes it a useful starting point for the design of miniature proteins that bind DNA and proteins and inhibit their interactions with high affinity and specificity, both in vitro and in mammalian cells and extracts. The utility of these molecules in a cellular context is complicated, however, by self-association: aPP typically forms a dimer at 10⁻⁶ M concentration.

This example illustrates the systematic isolation, quantification, and removal of two structural elements responsible for aPP dimerization and install a new element—a proline switch—that single-handedly repacks aPP's signature fold. The result is a monomeric and well-folded miniature protein that should accelerate the in vitro and in vivo applications of these molecules.

The structures of pancreatic fold proteins (PP) comprise an N-terminal type II polyproline helix that folds upon a C-terminal alpha helix to generate a stable, well-packed hydrophobic core. The first evidence of aPP self-association was the x-ray structure, which revealed an antiparallel dimer (FIG. 1A). FIG. 1A is a ribbon diagram of the aPP dimer highlighting pairs of Y7 (7), F20 (20), and Y21 (21) side chains. Molecular sieve chromatography further indicated that formation of the aPP dimer was pH- and temperature-dependent, with an equilibrium dissociation constant (K_{d}) between 400 pM and 5 micromolar.

Visual inspection of the aPP structure showed three potentially stabilizing interactions at the dimer interface (FIG. 1A). An intermolecular pi-stacking interaction between Y7 side chains was evident (FIG. 1B, which is a close-up of intermolecular network comprising Y7 and F20 from both monomers), with the orientation of Y7 defined by an intramolecular edge-to-face interaction with F20 (FIG. 1B); an intermolecular pi-stacking interaction between Y21 side chains was also observed (FIG. 1C, which is a close-up of intermolecular pi-stacking interaction between Y21 side chains). Comparison of the sequences of natural and designed PP-fold miniature proteins revealed that the self-associating molecules contained tyrosine at position 7, whereas non-associating pGolemi, p007, and PYY did not (FIG. 1D). In FIG. 1D, Alignment of natural and designed PP-fold proteins and variants prepared as part of this work. Residues that differ from wt aPP are shaded; those that differ between p007 and pGolemi are starred. The identities of the side chains at positions 20 and 21, however, did not correlate with self-association; most miniature proteins (including pGolemi) contained phenylalanine at position 20, and the residue at position 21 varied widely across the PP family.

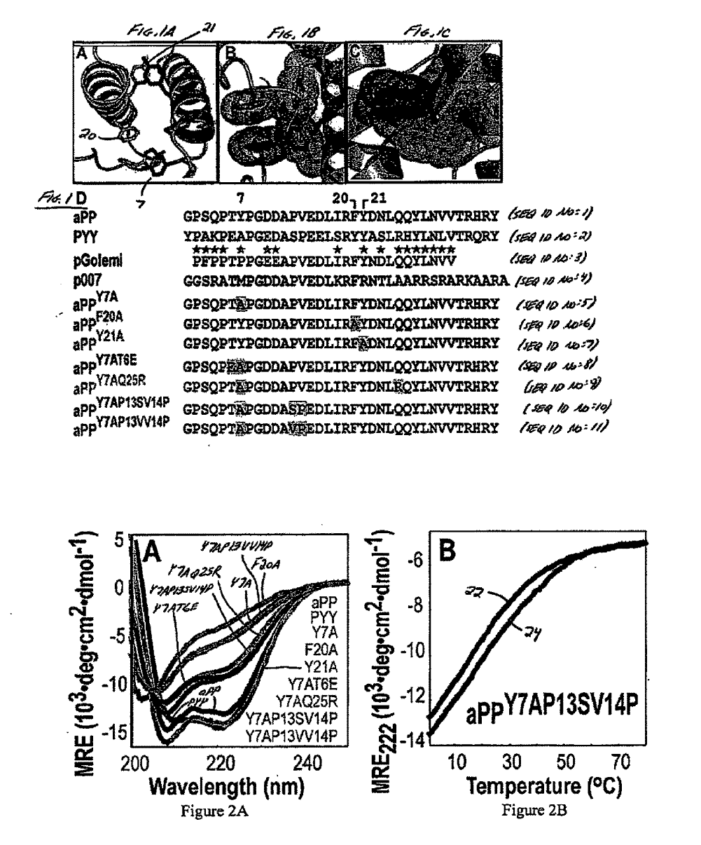

To determine the extent to which these residues contributed to aPP dimer stability, aPP variants were prepared containing alanine in place of each of the residues present at the dimer interface (aPP^{Y7A}, aPP^{F20A}, aPP^{Y21A}) and these were characterized using circular dichroism (CD) and analytical ultracentrifugation (AU). aPP^{Y7A} and aPP^{F20A} both assembled into tetramers, with K_{d} values of 3.9×10⁻¹² M³ and 7.6×10⁻¹¹ M³, respectively. Self-association of aPP^{Y7A} and aPP^{F20A} occurred only at high concentration, however: at 30 micromolar, more than 97% of the molecules remain monomeric. By contrast, aPP, with K_{d}=4.1×10⁻⁶ M, existed predominantly (>94%) in the dimer state at 30 micromolar. Although less prone to self-association than aPP, as monomers neither aPP^{Y7A} nor aPP^{F20A} assembled into the hairpin fold that characterizes PP-fold proteins, as judged by minimal negative ellipticity at 222 nm and a shift in the 208 nm minima to 205 nm (FIG. 2A, showing wavelength-dependent circular dichroism (CD) spectra of aPP variants). By contrast, aPP^{Y21A} formed a modestly more stable dimer than did aPP, as judged by analytical ultracentrifugation (K_{d}=1.3×10⁻⁶M) and CD (16,500 and 14,700 deg cm² dmol⁻¹ at 208 and 222 nm respectively). These data suggested that Y7 and F20 of aPP contributed to both dimer stability and maintenance of the characteristic aPP fold. Y21, although positioned at the dimer interface in the x-ray structure, contributed modestly to dimer stability.

Next, two strategies were explored for stabilizing and rebuilding the hairpin fold of monomeric aPP^{Y7A}. The first strategy builds on the observation that PYY possessed a stronger dipole moment (449 D) and a larger electrostatic stabilization energy (−10 kcal·mol⁻¹) than does aPP (430 D and −7 kcal·mol⁻¹, respectively). To evaluate whether macrodipole stabilization would increase the stability of monomeric (but otherwise poorly-folded) aPP^{Y7A}, a second set of variants was prepared containing two additional residues from PYY: a glutamic acid at position 6 (near the alpha helix N-terminus) and an arginine at position 25 (near the C-terminus). Sedimentation equilibrium experiments showed that neither aPP^{Y7AT6E} nor aPP^{Y7A,Q25R} was appreciably monomeric at 30 micromolar, with 45% and 46% of the solution forming tetramers respectively. The CD spectra of both molecules showed minima at 208 and 222 nm that were likely due to the large fraction of molecules assembled into well-folded tetramers at this concentration. These results indicate that macrodipole stabilization alone is insufficient to refold aPP^{Y7A}.

A more subtle difference between aPP and PYY is a proline residue whose location effectively demarcates the N-terminus of the alpha helix. This proline was highly conserved among PP-fold family members, however, its position varies between residue 13 and 14 (FIG. 1). By specifying where the alpha helix begins, this proline defined the relative orientation of the PPII and the alpha-helices and thus, the precise packing structure of the hydrophobic core. To determine whether this “proline switch” could improve the folding of poorly folded but monomeric aPP variants, two variants of aPP^{Y7A} were synthesized containing the sequences VP and SP at positions 13 and 14, in place of the natural PV sequence. APP^{Y7A,P13V,V14P} remained predominantly monomeric (90%) at 30 micromolar concentration, but lacked a well-defined conformation, as judged by CD. aPP^{Y7AP13SV14P}, however, displayed significant minima at both 208 (12,400 deg cm² dmol⁻¹) and 222 nm (9,700 deg cm² dmol⁻¹) (FIG. 2A). Temperature-dependent CD studies revealed that, like PYY, aPP^{Y7A,P13S,V14P} underwent a cooperative unfolding transition with a Tₘ of 20° C. (FIG. 2B). FIG. 2B shows temperature-dependent change in the ellipticity at 222 nm of aPP^{Y7A,P13S,V14P} as the temperature is raised (24) and then lowered (22). All spectra were acquired at 30 micromolar concentration in Tris-Cl buffer (25 mM Tris, 50 mM NaCl, (pH 8.0)). AU experiments were also conducted at temperatures from 5° C. to 30° C. (5° C. increments) to fully characterize how the extent of self-association varies with temperature. At 25° C., the data for aPPY^{7A,P13S,V14P} fit a monomer/dimer/tetramer model with K_{D} values of 5.3×10⁴ M and 6.8×10⁻¹²M³ respectively, which corresponded to 90% monomer, 9% dimer, and 1% tetramer at 30 micromolar.

In conclusion, some, but not all, side chains located at the aPP dimer interface contributed significantly to dimer stability. Although substitutions at these positions reduced dimer stability at the expense of tertiary structure, insights gleaned from comparisons among natural and designed PP-fold family members identified a key “proline-switch” that restored the signature aPP fold. The result is a well-folded miniature protein that was monomeric at concentrations above that where aPP-derived miniature proteins associate with their macromolecular targets.

Additional information in regards to the above follows. Avian pancreatic polypeptide (aPP) was purchased from American Peptide Company Inc. (cat #46-8-25) as a purified lyophilized powder. All other peptides were synthesized on a Symphony® multi-channel solid phase synthesizer (Protein Technologies, Inc., Tucson, Ariz.) using Fmoc-protected amino acid monomers and NovaSyn TGR resin (cat. 01-64-0060) from Novabiochem (San Diego, Calif.). N,N-Dimethylformamide, N-methyl morpholine, piperidine, and trifluoroacetic acid were purchased from American Bioanalytical (Natick, Mass.). Mass spectra were acquired with an Applied Biosystems Voyager-DE-Pro matrix-assisted laser desorption/ionization time-of-flight (MALDI-TOF) mass spectrometer (Foster City, Calif.). Reverse-phase HPLC was performed using a Rainin Dynamax HPLC with a Vydac analytical C8 column (300 angstrom silica, 5 micrometer particle size, 4.6 mm 5 150 mm) and a Grace Vydac C8 preparative scale column (300 angstrom silica, 5 micrometer particle size, 22 mm×150 mm), and water/acetonitrile gradients containing 0.1% TFA. Circular dichroism (CD) spectra were acquired with a Jasco J-810 Spectropolarimeter (Jasco, Tokyo, Japan) equipped with a Peltier temperature control module. Analytical ultracentrifugation experiments were performed using a Beckman Coulter Proteome Lab XL-I Protein Characterization System equipped with an AN 60-Ti 4-hole rotor and six-channel carbon-epoxy composite centerpieces (Beckman, Fullerton, Calif.).

All peptides were synthesized using standard solid-phase Fmoc chemistry (25 micromole scale) on an automated peptide synthesizer and contained a free amine at the N-terminus and a carboxamide at the C-terminus. Crude peptides were purified by reverse phase HPLC and identified by MALDI-TOF mass spectrometry. Peptide purity was verified by reinjection of a small aliquot of the purified sample on an analytical C8 column. Following purification, the peptide was lyophilized, resuspended in water, and dialyzed against water to remove residual TFA salts using Floatalyzers® (Spectrum Labs, cat #235026) with a 500 Da MWCO. Peptide solutions were then divided into aliquots and stored at −20° C.

MALDI mass spectra were obtained using a 1:1 ratio of peptide to alpha-cyano-4-hydroxycinnaminic acid matrix (Table 1).

TABLE 1

| | | Calcu- | |
|---|---|---|---|
| | | lated | |
| Peptide | | Mass | Observed |
| Name | Peptide Sequence | (Da) | Mass (Da) |
| PYY | YPAKPEAPGEDASPEELSR | 4241 | 4241 |
| | YYASLRHYLNLVTRQRY | | |
| | (SEQ ID NO: 5) | | |
| PGolemi | PFPPTPPGEEAPVEDLIRF | 3459 | 3466 |
| | YNDLQQYLNVV | | |
| | (SEQ ID NO: 9) | | |
| p007 | GGSRATMPGDDAPVEDLLKR | 4168 | 4172 |
| | FRNTLAARRSRARKAARAA | | |
| | (SEQ ID NO: 10) | | |
| aPP^{Y7A} | GPSQPTAPGDDAPVEDLIRF | 4146 | 4145 |
| | YDNLQQYLNVVTRHRY | | |
| | (SEQ ID NO: 11) | | |
| aPP^{F20A} | GPSQPTYPGDDAPVEDLIRA | 4162 | 4158 |
| | YDNLQQYLNVVTRHRY | | |
| | (SEQ ID NO: 12) | | |
| aPP^{Y21A} | GPSQPTYPGDDAPVEDLIRF | 4146 | 4145 |
| | ADNLQQYLNVVTRHRY | | |
| | (SEQ ID NO: 13) | | |
| aPP^{Y7AT}_{6E} | GPSQPEAPGDDAPVEDLIRF | 4174 | 4176 |
| | YDNLQQYLNVVTRHRY | | |
| | (SEQ ID NO: 14) | | |
| aPP^{Y7AQ}_{25R} | GPSQPTAPGDDAPVEDLIRF | 4174 | 4176 |
| | YDNLRQYLNVVTRHRY | | |
| | (SEQ ID NO: 15) | | |
| aPP^{Y7AP}_{13SV14P} | GPSQPTAPGDDASPEDLIRF | 4134 | 4131 |
| | YDNLQQYLNVVTRHRY | | |
| | (SEQ ID NO: 16) | | |
| aPP^{Y7AP}_{13VV14P} | GPSQPTAPGDDAVPEDLIRF | 4146 | 4141 |
| | YDNLQQYLNVVTRHRY | | |
| | (SEQ ID NO: 17) | | |

The CD spectra of each peptide (30 micromolar) were acquired in Tris buffer (25 mM Tris base, 50 mM NaCl, (pH 8.0)) at 25° C. in a 2 mm cell. Samples were scanned between 200 and 260 nm, with signal sampling every 0.5 nm, and three successive scans were averaged. Molar residue ellipticity values were calculated from the equation MRE=(Θₛₐₘₚₗₑ−Θ_{buffer})/(L×c×n×1000), where Θ is the observed signal in millidegrees, L is the length of the cuvette in cm, c is the concentration of peptide in dmol cm⁻³ and n is the number of amino acids in the molecule.

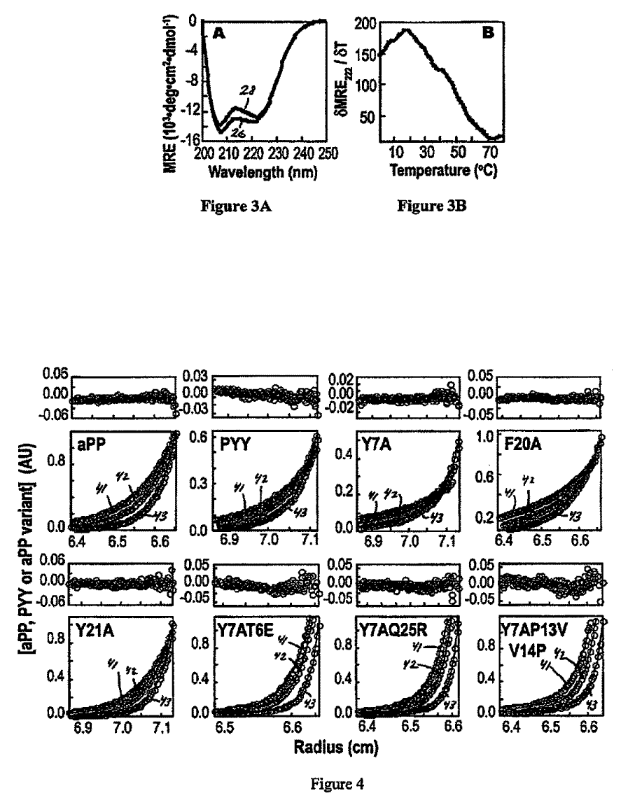

Temperature-dependent CD spectra of aPP^{Y7AP13SV14P} (30 micromolar) were acquired in Dulbecco's PBS (1 mM KH₂PO₄, 155 mM NaCl, 3 mM Na₂HPO₄, (pH 7.4)) in a 2 mm cell. The signal at 222 nm (MRE₂₂₂) was monitored between 1° C. and 80° C. using the variable temperature module provided with the instrument. Data were collected with a 0.5° C. data pitch, 5 s delay time, 20° C./hour temperature slope, 4 s response, and 1 nm bandwidth. CD spectra were obtained as the temperature was raised and when the temperature was lowered to establish the reversibility of the folding transition. Additionally, a full wavelength scan was obtained before and after the thermal melt; an overlay of the two spectra is shown in FIG. 3A. FIG. 3 shows a CD analysis of aPP^{Y7A,P13S,V14P} (30 micromolar). FIG. 3A shows wavelength-dependent CD spectra acquired at 5° C. in PBS buffer either immediately prior to (26) or following (28) temperature-dependent CD scans shown in FIG. 2B. Mean residue ellipticity values for the temperature dependent CD spectrum were calculated from the equation MRE=Θ₂₂₂ nm/(L×c×n×1000), where Θ is observed signal at 222 nm (mdeg), L is the length of, the cuvette (cm), c is the concentration of peptide (dmol/cm³) and n is the number of amino acids in the molecule. The first derivatives of the temperature-dependent CD spectra are shown in FIG. 3B, which is a plot of δMRE₂₂₂/δT (units of deg cm² dmol⁻¹ K⁻¹). The Tₘ value reported in the main text corresponds to the temperature at which ∂MRE₂₂₂/∂T is a maximum.

Analytical ultracentrifugation experiments were performed using peptide solutions prepared in Tris.Cl buffer (25 mM Tris base, 50 mM NaCl, (pH 8.0)) at the appropriate concentrations (5 micromolar to 200 micromolar). Samples were then centrifuged to equilibrium at 25° C. at 42,000, 50,000, and 60,000 rpm. Data were collected with a 0.001 cm step size and successive scans were initiated at 2 h intervals. Samples were judged to have reached equilibrium when the radial concentration gradient remained unchanged over three successive scans using the Match module of Heteroanalysis software v1.1.19. The solvent density (ρ, rho) and partial specific volume ( ν̅, nu) were calculated using the Sedimentation Interpretation Program “Sednterp” software v1.08.

The data for each peptide (multiple concentrations at three speeds) was first fit globally to an equation describing the sedimentation of an ideal species of molecular weight Mₙ (Eq. 1) using Heteroanalysis software v1.1.19:

$$C ( r ) = C ( {r}^{0} ) exp \begin{matrix}( 1 - v ρ ) {M}_{n} {ω}^{2} \\ 2 RT\end{matrix} ( {r}^{2} - {r}_{0}^{2} ) , \qquad Equation 1.$$

where C represents the concentration of the sedimenting species at distances r and r₀ cm from the center of rotation; ν (nu) is the partial specific volume of the sedimenting species in cc/g; ρ (rho) is the density of the supporting buffer in g/cc; ω (omega) is the angular velocity of the rotor (radians/s); Mₙ is the “molar” molecular weight of sedimenting species (g/mol); R is the universal gas constant (8.315×10⁷ ergs K⁻¹ mol⁻¹) and T is the temperature in degrees Kelvin. In all cases, the best fits to this ideal model were characterized by MWₐᵥₑ values that were significantly higher than MW_{calc}, indicating that each peptide self-associating in the concentration range studied.

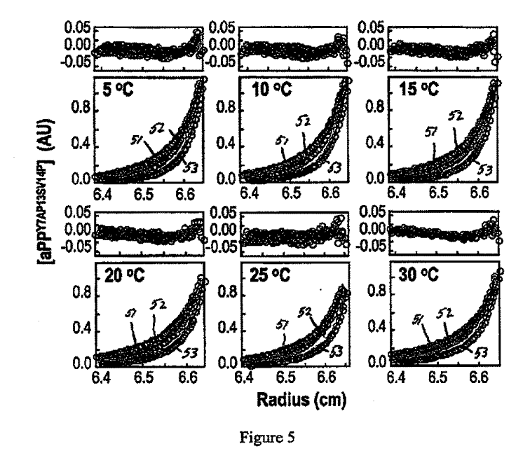

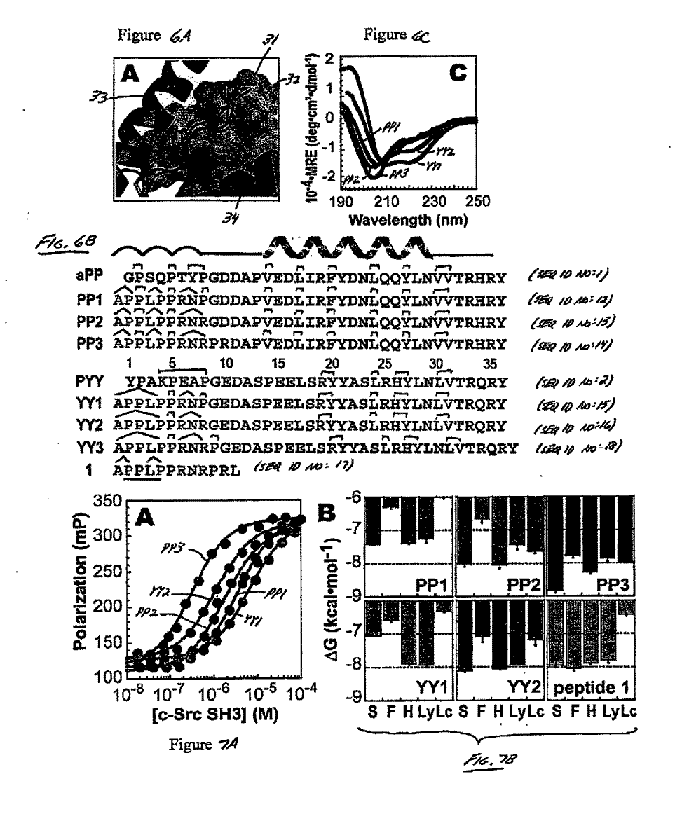

In order to quantify this self-association, the data for each peptide was next fit globally to a monomer-n-mer equilibrium model using Eq. 2:

$${C ( r )}_{tot} = {C ( {r}^{0} )}_{monomer} exp \frac{( 1 - \overline{v} ρ ) {M}_{n} {ω}^{2}}{2 RT} + {C ( {r}^{0} )}_{n \text{-} mer} exp \begin{matrix}( 1 - \overline{v} ρ ) {nM}_{n} {ω}^{2} \\ 2 RT\end{matrix} ( {r}^{2} - {r}_{0}^{2} ) . \qquad Equation 2$$

where n is the stoichiometry of the self-associated complex. Errors were calculated for n and lnK_{A} using f-statistics to two standard deviations. Best fits of the data to Eq. 2 are shown in FIG. 4; a summary of the fitted parameters are listed in Table 2.

In particular, FIG. 4 shows sedimentation equilibrium analysis of aPP (14 micromolar), PYY (125 micromolar), aPP^{Y7A} (45 micromolar), aPP^{F20A} (12.5 micromolar), aPP^{Y21A} (60 micromolar), aPP^{Y7AT6E} (90 micromolar), micromolar), and aPP^{Y7AP13VV14P} (160 micromolar). Samples were prepared in PBS buffer and centrifuged at speeds of 42,000 (41), 50,000 (42), or 60,000 RPM (43) at 25° C. Experimental data are shown as points; lines indicate the best fit to a monomer-n-mer model described by Eq. 2.

TABLE 2

| | MWₐᵥₑ | | | | % monomer at |
|---|---|---|---|---|---|
| Peptide | (Da) | n^{a} | lnK_{A}^{a} | K_{d}^{b} | 30 μM |
| aPP | 6619 | 1.92 | when n = 2 12.41 | 4.1 × 10⁻⁶ M | 23 |
| | | (1.79, | (12.16, 12.68) | | |
| | | 2.06) | | | |
| PYY | 5757 | 2.51 | when n = 2 7.09 | 8.3 × 10⁻⁴ M | 94 |
| | | (2.28, | (6.95, 7.23) | | |
| | | 2.76) | | | |
| aPP^{Y7A} | 4818 | 3.77 | when n = 4 26.28 | 3.9 × 10⁻¹² | 98 |
| | | (3.62, | (26.13, 26.42) | M³ | |
| | | 3.93) | | | |
| aPP^{F20A} | 4351 | 3.87 | when n = 4 23.29 | 7.6 × 10⁻¹¹ | 100 |
| | | (3.85, | (23.16, 23.42) | M³ | |
| | | 3.88) | | | |
| aPP^{Y21A} | 7676 | 1.86 | when n = 2 13.57 | 1.3 × 10⁻⁶ M | 14 |
| | | (1.84, | (12.95. 14.16) | | |
| | | 1.88) | | | |
| aPP^{Y7AT6E} | 10480 | 3.88 | when n = 4 29.26 | 2.0 × 10⁻¹³ | 79 |
| | | (3.68, | (29.04, 29.49) | M³ | |
| | | 4.09) | | | |
| aPP^{Y7AQ25R} | 10844 | 4.06 | when n = 4 29.34 | 1.8 × 10⁻¹³ | 78 |
| | | (3.88, | (29.15, 29.53) | M³ | |
| | | 4.24) | | | |
| aPP^{Y7AP13VV14P} | 9961 | 4.48 | when n = 4 27.92 | 7.5 × 10⁻¹³ | 90 |
| | | (4.21, | (27.68, 28.16) | M³ | |
| | | 4.76) | | | |

^{a}Lower and upper error values calculated using f-statistics are shown in parentheses.
^{b}K_{D} values calculated from ln K_{A}.

aPP^{Y7AP13SV14P} samples were prepared in Dulbecco's PBS buffer (as described above) at concentrations of 45, 90, and 180 micromolar. Sedimentation equilibrium experiments were performed as described above and monitored at 280 nm. Samples were centrifuged to equilibrium at three speeds (42,000, 50,000, and 60,000 rpm) at 5, 10, 15, 20, 25, and 30° C. The rotor containing the samples was agitated manually between temperature runs to redistribute the sample along the length of the cell and checked by an initial scan.

The sedimentation data obtained for aPP^{Y7AP13SV14P} at each temperature was globally fit to Eq. 2 as described above. Not surprisingly, n varied with temperature, increasing from 2.5 at 5° C. to 3.6 at 30° C. Therefore, the data from within each temperature set was then globally fit to a monomer-n-mer-m-mer model. Based on the self-association trends of the other aPP variants, in which the folded peptides best fit to a monomer-dimer model and the unfolded peptides fit to a monomer-tetramer model, the values for n and m were fixed to 2 and 4 respectively. Data fit with these parameters and the resulting residuals are presented in FIG. 5 and a summary of the fitted parameter results are given in Table 3. In particular, FIG. 5 shows temperature-dependent sedimentation equilibrium of aPPY^{7AP13SV14P}. Samples were prepared in PBS buffer (90 micromolar) and centrifuged to equilibrium at speeds of 42,000 (51), 50,000 (52), and 60,000 (53) rpm. Experimental data are shown as points; lines indicate a fit to a monomer-dimer-tetramer model as described with resulting residuals displayed as well.

TABLE 3

| Temperature | ln K_{A} (n) | ln K_{A} (m) | % monomer^{a} |
|---|---|---|---|
| 5° C. | 8.9 (8.4, 9.3)^{b} | 25.7 (25.3, 26.0) | 74 |
| 10° C. | 8.4 (7.9, 8.8) | 25.3 (25.0, 25.5) | 82 |
| 15° C. | 8.2 (7.7, 8.6) | 25.6 (25.4, 25.8) | 84 |
| 20° C. | 7.5 (6.4, 8.2) | 25.0 (24.8, 25.2) | 90 |
| 25° C. | 7.5 (6.4, 8.2) | 25.7 (25.4, 26.0) | 90 |
| 30° C. | 5.6 (ND) | 24.4 (24.2, 24.5) | 98 |

^{a}At 30 micromolar total peptide concentration
^{b}Lower and upper error values calculated using f-statistics are shown in parentheses.

### Example 2

There is considerable interest in encodable molecules that regulate intracellular protein circuitry and/or activity, ideally with high levels of specificity. One class of tightly regulated signaling proteins, the Src family kinases, contain a catalytic kinase domain and regulatory Src homology 2 (SH2) and Src homology 3 (SH3) domains. Src kinases are maintained in an inactive state by virtue of intramolecular interactions between the SH2 domain and a phosphotyrosine sequence in the C-terminal tail and between the SH3 domain and a proline-rich sequence in the SH2-kinase linker. Src kinases can be activated by ligands that disrupt either or both of these interactions, and different activation modes may lead to different downstream signaling events. Thus, encodable molecules that activate select Src family kinases in well-defined ways could complement selective Src kinase inhibitors to unravel the roles of specific family members in cell signaling events. This example describes a set of miniature proteins that recognize SH3 domains from distinct Src-family kinases with high affinity; two of them activate Hck kinase with potencies that rival HIV Nef, which activates Hck kinase in vivo.

The NMR structure of the c-Src SH3 domain in complex with peptide 1 (SEQ ID NO: 17) guided design of the molecules studied here (FIG. 6A). FIG. 6A shows the structure of 1 (APP12, 31) in complex with c-Src SH3 (32) (PDB 1QWE), superimposed with aPP (33, PDB 1PPT) and PYY (34, PDB 1RU5). This structure shows 1 bound as a PPII helix with the side chains of P₁, L₃ and P₄ nestled into grooves of the SH3 domain surface. The side chain of R₆ anchored the peptide in a class II orientation and that of N₇ provided additional affinity. Substitution of these five residues for analogous residues within aPP, the PP-fold protein used previously for miniature protein design, led to PP1 (FIG. 6B).

In this figure, residues that contribute directly to c-Src SH3 recognition are indicated by angle brackets; those that contribute to aPP/PYY folding are indicated by square brackets. The PxxP core epitope is underlined. Inclusion of one (R₈) or three (R₈-R₁₀) additional residues from 1 at the C-terminus of the motif led to PP2 and PP3. To complement the aPP-based designs, a pair of miniature proteins were also prepared (YY1 and YY2) based on the aPP ortholog PYY. PYY also displayed a characteristic PP-fold, but its variants were more soluble and less prone to dimerization than aPP variants.

First, the secondary structures and thermal stabilities of the aPP and PYY-based designs were compared using circular dichroism (CD) spectroscopy (FIG. 6C). The CD spectra of the aPP-based molecules (PP1, PP2 and PP3) showed little ellipticity at 208 and 222 nm, indicating little alpha-helical secondary structure under these conditions. By contrast, the CD spectra of PYY-based YY1 and YY2 showed significant signals at these wavelengths, with mean residue ellipticities at 222 nm (MRE₂₂₂) of −1.4×10⁴ and −1.0×10⁴ deg cm² dmol⁻¹, respectively. Temperature dependent CD studies indicated that YY1 and YY2 underwent cooperative melting transitions with midpoints (T_{M}) of ˜25° C. and ˜50° C., respectively. The spectral signature of YY1 was essentially identical to that of wild type PYY (MRE₂₂₂ of −1.6×10⁴ deg cm² mol⁻¹, Tₘ˜50° C.; unpublished data), which suggested that it retained the characteristic PP-fold structure. YY1, like PYY, was a monomer at low micromolar concentrations (K_{d}=180 micromolar).

The affinity of each miniature protein for the c-Src SH3 domain was determined using fluorescently labeled miniature proteins and a direct polarization assay (FIG. 7A). In this figure, the fluorescence polarization analysis of the binding of c-Src SH3 by miniature proteins (50 nM) is shown. Peptide 1, whose affinity for c-Src SH3 was optimized by phage display, bound c-Src SH3 well under these conditions, with an equilibrium affinity (K_{d}=1.5±0.1 micromolar) analogous to that reported (K_{d}=1.2 micromolar). Although PP1 and PP2 bound c-Src SH3 with affinities close to that of 1 (K_{d}=3.68±0.06 micromolar and 1.3±0.2 micromolar, respectively), PP3 bound significantly better (K_{d}=350±40 nM). It is notable that the main difference between PP3 and PP1/PP2 is the presence of P₉-R₁₀. Neither 1 nor any miniature protein studied here bound detectably (K_{d}>20 micromolar) to c-Src SH3^{P133L}, a variant containing a mutation in the core binding groove that disrupts c-Src activity in S. pombe. The affinities of YY1 and YY2 for c-Src SH3 were essentially identical to those of the corresponding aPP-based designs despite differences in intrinsic secondary structure and virtually identical contact surfaces. Taken together, these results indicate that both classes of miniature protein ligands have potential as encodable ligands for SH3 domains. Although miniature protein structure often contributes significantly to binding affinity, in this case it offered no measurable advantage.

To evaluate whether miniature protein structure contributed to binding specificity, the relative affinity of each miniature protein for SH3 domains within (Fyn, Hck, Lyn, Lck) and outside (Ab1, Nck1, Grb2, Abp1) the Src kinase family as determined. None of the miniature proteins, nor peptide 1, bound well to any non-Src family SH3 domain tested (K_{d}>20 micromolar; data not shown). Peptide 1 showed little specificity within the Src family, binding well to the domains from Fyn, Hck, and Lyn (0.87 micromolar <K_{d}<2.0 micromolar) and poorly (K_{d}>20 micromolar) to Lck SH3 (FIG. 7B). This figure shows a comparison of binding free energies for complexes between aPP- and PYY-based miniature proteins and the SH3 domains of c-Src (S), Fyn (F), Hck (H), Lyn (Ly) and Lck (Lc). Values shown represent the average of at least three trials±standard error. By contrast, all of the miniature proteins, but especially PP3 and YY1, showed significantly greater, and different, specificity. PP3 preferred the Src SH3 domain to all others tested (ΔΔG=0.6 to 1.0 kcal mol⁻¹), whereas YY1 preferred the SH3 domains of Hck and Lyn over Src (ΔΔG=0.8 and 0.9 kcal mol⁻¹, respectively). Notably, YY1 and PP1 displayed different preferences despite the presence of identical sequence over nine N-terminal residues; the same is true for YY2 and PP2. This pattern suggested that SH3 domain specificity, even among close family members, can be fine-tuned by miniature protein sequence and architecture. A similar result can be seen for YY3, as shown in FIG. 7C.

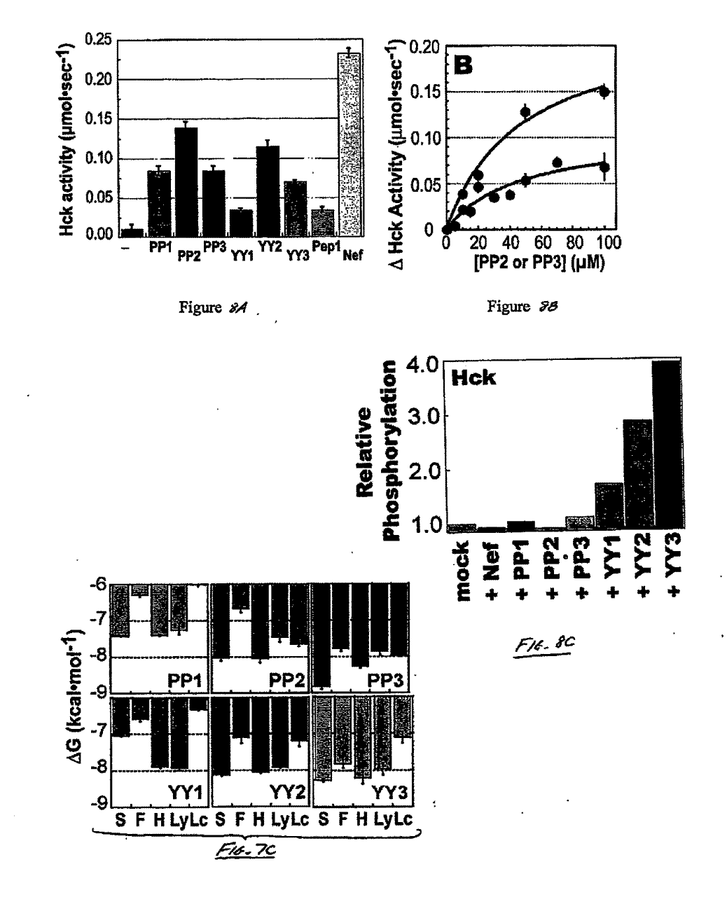

SH3 domains regulate the activity of Src family kinases through interaction with an internal proline-rich region that locks the kinase into a catalytically repressed state. Ligands such as HIV Nef and H. saimiri Tip which block this interaction up-regulate kinase activity. To evaluate whether the miniature proteins studied here could function as encodable activators of a Src family kinase, their effect on Hck activity was monitored using an assay that couples ATP hydrolysis to NADH oxidation. Hck was chosen rather than Src because of the availability of Nef as a potent positive control. As expected, Nef was a potent Hck activator, increasing kinase activity 21-fold at 50 micromolar concentration (˜200 K_{d}). By contrast, at the same concentration peptide 1 was a modest Hck activator, increasing Hck activity 3-fold. All miniature proteins except YY1 were significantly more potent activators than Peptide 1 (FIG. 8A). Values of K_{act} determined for the most active molecules PP2 and PP3 were 48±13 micromolar and 48±22 micromolar, respectively (FIG. 8B). Specifically, FIG. 8A shows the activity of Hck kinase in the presence or absence of 50 micromolar miniature protein, Peptide 1 (FIG. 8A), or Nef or PP2 and PP3 at the concentrations indicated (FIG. 8B). Values shown represent the average of three determinations±standard error. FIG. 8C illustrates relative phosphorylation for these compounds, illustrating that YY3 activates Hck in vitro. In addition, PPY-based miniature proteins, such as these, may also activate Hck kinase activity and/or Src activity in live cells, such as in NIH 3T3 fibroblasts.

These values were modestly higher than the published value for Nef (K_{act}=18.0 micromolar), and approached the values reported for potent but non-encodable peptoids. Surprisingly, Hck activation correlated with neither SH3 domain affinity nor Hck specificity—PP2, PP3, YY1, YY2 and 1 all bound Hck SH3 with comparable affinities in vitro, yet YY1 did not activate; PP1 and PP3 were equipotent activators yet PP1 bound poorly. These differences could result from differences in affinity for full-length kinases or from differences in binding mode that correspond to alternative activation levels.

Additional information in regards to the above follows. Miniature proteins PP1, PP2, PP3, YY1, YY2, Peptide 1 (or APP12) and the Hck substrate peptide (SEQ ID NO: 19) were synthesized using standard solid-phase Fmoc chemistry (25 micromole scale) and a Symphony® multi-channel solid phase synthesizer (Protein Technologies, Inc., Tuscon, Ariz.). All alpha-amino acids and NovaSyn TGR resin (cat. 01-64-0060) was purchased from Novabiochem. All miniature proteins were synthesized to contain free amines at their N-termini and carboxamides at their C-termini. With the exception of Peptide 1, all miniature proteins or peptides employed in fluorescence polarization (FP) assays contained an additional cysteine residue at the C-terminus which was labeled as described below. The fluorescently labeled analog of Peptide 1 (^{Flu}1) contained an additional cys-gly-gly sequence at the N-terminus and lacked the C-terminal leu.

Crude peptides were purified by reverse-phase HPLC using a Waters instrument equipped with a 1525EF binary pump and 2996 photo-diode array detector and was operated using the Empower software suite. Samples were purified using Grace Vydac C8 or C18 preparative scale columns (300 angstrom silica, 10 micrometer particle size, 22 mm×250 mm) and water/acetonitrile gradients containing 0.1% TFA. Peptide identity was verified by mass spectrometry using an Applied Biosystems Voyager-DE Pro MALDI-TOF mass spectrometer (Foster City, Calif.). Peptide purity was confirmed by re-injection of a purified sample on a Vydac analytical C8 column (300 angstrom silica, 5 micrometer particle size, 4.6 mm×150 mm). Peptide concentration and identity were also confirmed by amino acid analysis at the HHMI Biopolymer/Keck Foundation Biotechnology Resource Laboratory at the Yale University School of Medicine. Once purified, the miniature proteins were lyophilized to dryness, brought up in water and stored at −20° C.

The miniature proteins or peptides were labeled using 5-iodoacetamidofluorescein (Molecular Probes). A typical reaction contained about 5 mg HPLC purified miniature protein or peptide and 10 equivalents 5-iodoacetamidofluorescein in a 1:1 mixture of 10 mM sodium phosphate (pH 8) and DMSO (dimethylsulfoxide). Labeling reactions were incubated with rotation for 3 h at room temperature or about 10 h at 4° C. Labeled product was purified by reverse-phase HPLC and its identity verified by MALDI-TOF and amino acid analysis as described above. After purification, all fluorescently labeled miniature proteins were lyophilized, brought up in water and stored at −20° C. protected from light.

TABLE 4

| | Calculated Mass (Da) | Observed Mass (Da) |
|---|---|---|
| Peptide 1 | 1383.7 | 1383.7 |
| PP1 | 4350.8 | 4354.5 |
| PP2 | 4410.0 | 4417.0 |
| PP3 | 4491.2 | 4495.0 |
| YY1 | 4327.8 | 4328.6 |
| YY2 | 4386.9 | 4386.8 |
| ^{Flu}1 | 1874 | 1880.38 |
| PP1^{Flu} | 4839.36 | 4846.5 |
| PP2^{Flu} | 4901.46 | 4910.0 |
| PP3^{Flu} | 4982.66 | 4993.7 |
| YY1^{Flu} | 4818.4 | 4826.1 |
| YY2^{Flu} | 4878.4 | 4882.2 |
| Hck substrate | 1856.06 | 1856.5 |

Protein expression and purification was as follows. The pGEX-4T-1 plasmids used to over-express the SH3 domain-GST fusion proteins of human Hck (residues 77-137 of full-length kinase), human Fyn (residues 81-142) and human Lyn (residues 62-122) were obtained from Dr. Wendell Lim (UCSF); the pGEX-2T plasmid used to over-express the chicken c-Src (residues 81-140) SH3 domain-GST fusion protein was obtained from Dr. Stuart Schreiber (Broad Institute); and the pGEX-3Xb plasmid used to express the human Lck (residues 62-128) SH3 domain-GST fusion was from Dr. Philip Cole (Johns Hopkins). The Hck and Csk baculovirus and the Nef-GST fusion pGEX construct were kind gifts from Dr. W. Todd Miller (SUNY-Stonybrook). The c-Src SH3^{P133L} variant was prepared using QuikChange® site-directed mutagenesis of the c-Src construct mentioned above; its sequence was verified by DNA sequencing at the W.M. Keck Foundation Biotechnology Resource Laboratory at the Yale University School of Medicine.

The plasmids were transformed into BL21(DE3) E. coli cells (Stratagene) and a single colony was used to inoculate a 500 mL culture in LB media supplemented with 50 micrograms/mL carbenicillin. The culture was incubated at 37° C. with shaking at about 250 rpm until the solution reached an optical density at 600 nm (OD₆₀₀) of about 0.8 absorbance units. Isopropyl beta-D-galactoside (IPTG) was added to a final concentration of 0.5 mM and the incubation continued for another about 5 h at 37° C. with shaking. Cells were harvested by centrifugation at 2,700×g and the resulting cell pellet was flash-frozen. The pellet was then thawed, resuspended in 30 mL PBS (1 mM KH₂PO₄, 155 mM NaCl, 3 mM Na₂HPO₄ (pH 7.4)) and the cells lysed (2 passes) using a French® Pressure Cell Press (SLM Instruments, Inc) with the Sim Aminco french pressure cell (cat. # FA-073). Phenylmethylsulfonylfluoride (PMSF, final concentration 1 mM) was added to the suspension immediately before lysis. The cell lysate was cleared by centrifugation at 21,000×g and the supernatant incubated with pre-equilibrated Glutathione Sepharose 4B (Amersham Pharmacia; 4 mL resin per 500 mL cell culture) overnight at 4° C. with end-over-end rotation. The fusion protein was eluted from the resin using 4×1 mL of Elution Buffer (10 mM glutathione, 50 mM Tris (pH 8.0)). The purity of the fractions was assessed by SDS PAGE; pure fractions were pooled and the resulting solution was dialyzed at 4° C. using Slide-a-Lyzers® dialysis cassettes (cat. # 66425, Pierce) against PBS buffer supplemented with dithiothreitol (DTT, 1 mM final concentration). Stocks of SH3 domain-GST fusion proteins used in fluorescence polarization assays (100-200 micromolar) were separated into aliquots, flash-frozen and stored at −20° C. The concentrations of all SH3 domain-GST fusion proteins were determined by amino acid analysis as described above.

The HIV Nef protein employed as a positive control for the Hck activation assay was expressed in BL21 (DE3) cells as a GST fusion. Transformed cells were grown in LB supplemented with 50 micrograms/mL carbenicillin (1 L) at 37° C. to OD₆₀₀=0.7, induced at 25° C. with 0.5 mM IPTG and incubated for approximately 22 h. The cells were pelleted (4000×g, 10 min), resuspended in 30 mL PBS (1 mM KH₂PO₄, 155 mM NaCl, 3 mM Na₂HPO₄ (pH 7.4)) supplemented with 1 mM DTT, 1 mM PMSF and 1 tablet Roche EDTA-free protease inhibitor (cat #11873580001, 1 tablet per 30 mL or 1 mini-tablet per 10 mL volume) and lysed using a French Press as described above. The cell lysate was cleared by centrifugation (10,000×g, 30 min) and the supernatant was incubated at 4° C. with Glutathione Sepharose 4B resin (2 mL per 1 L cell culture, 1 h) pre-equilibrated with PBS. The resin was washed with PBS containing 1 mM DTT (20 mL) and the fusion protein cleaved while resin-bound with 100 U thrombin (Amersham Pharmacia) for 16 h according to the manufacturer's protocol. Nef was eluted with PBS (1 mL) and the buffer was exchanged using a NAP-25 column (cat #17-0852-01, Amersham Pharmacia) equilibrated with Nef IE Buffer (20 mM Tris (pH 7.0), 50 mM KCl, 1 mM PMSF, 1 mM DTT). To separate Nef from thrombin, the dialyzed sample (3.5 mL) was loaded onto a 5 mL HiTrap Q XL ion exchange column (Amersham Pharmacia) that had been pre-equilibrated with Nef IE Buffer and eluted with a linear NaCl gradient (0-1.0 M) using the ÄKTA™ FPLC with Unicorn 5.01 software (Amersham Biosciences Corp.). Fractions containing Nef were identified by SDS-PAGE, pooled, the buffer exchanged into Nef Freeze Buffer (20 mM Tris, pH 8.5, 50 mM KCl, 1 mM DTT, 1 mM PMSF, 10% glycerol) as described above, separated into aliquots and stored at −20° C.

Down-regulated Hck kinase (containing an N-terminal His₆ tag) has been co-expressed previously with Csk in Sf9 cells. HighFive cells (Invitrogen) were used, which are reported to support higher protein expression levels. The procedure described below typically produced 0.5 mg purified recombinant protein from a 50 mL HighFive culture. All buffers used for Hck purification contained 1 mM PMSF and Roche EDTA-free protease inhibitor cocktail tablets. HighFive cells were grown in suspension (50 mL) in Express Five serum-free media (cat #10486-025, Gibco) supplemented with 20 mM glutamine and 10 micrograms/mL Gentamycin to a density of 2.0×10⁶ cells/mL and infected with Hck and Csk baculovirus at multiplicity of infection of 5. Cells were harvested by centrifugation after 48 h (800×g, 10 min) and stored at −20° C. The cell pellet was thawed and resuspended in 7.5 mL HisLysis buffer (20 mM Tris (pH 8.5), 150 mM KCl, 0.01% beta-mercaptoethanol, 0.04% NP40, 10 mM imidazole, 5% glycerol). Triton X-100 (1%) was added and the cells lysed by end-over-end rotation for 15 minutes at 4° C. The lysate was cleared by centrifugation (100,000×g) for 1 h and the supernatant added to 0.5 mL Ni-NTA resin, washed with 20 mL HisBuffer A (20 mM Tris, 500 mM KCl, 0.04% beta-mercaptoethanol, 0.01% NP40, 20 mM imidazole, 5% glycerol (pH 8.5) and incubated with end-over-end rotation for 1 h at 4° C. The resin was transferred to an empty PD10 column, drained and washed successively with 10 mL of HisBuffer A, 2.5 mL HisBuffer B (20 mM Tris, 1M KCl, 0.04% beta-mercaptoethanol, 0.01% NP40, 5% glycerol (pH 8.5)), 1 mL HisBuffer A and the protein eluted with 8×1 mL washes with HisElution buffer (20 mM Tris, 150 mM KCl, 0.04% beta-mercaptoethanol, 0.01% NP40, 100 mM imidazole, 5% glycerol (pH 8.5)). Each wash was incubated for 1 min with the resin before collection. The fractions containing Hck were identified by SDS-PAGE, pooled, concentrated (Amicon Ultra-4 Centrifugal Filter Device, 10 k MWCO, cat #UFC801024, Millipore) to 1 mL (approximately 0.6 mg/mL) and dialyzed against Hck Freeze Buffer (20 mM Tris (pH 8.5), 50 mM NaCl, 3 mM DTT, 10% glycerol, 2×500 mL). The enzyme was aliquoted, flash-frozen and stored at −20° C.

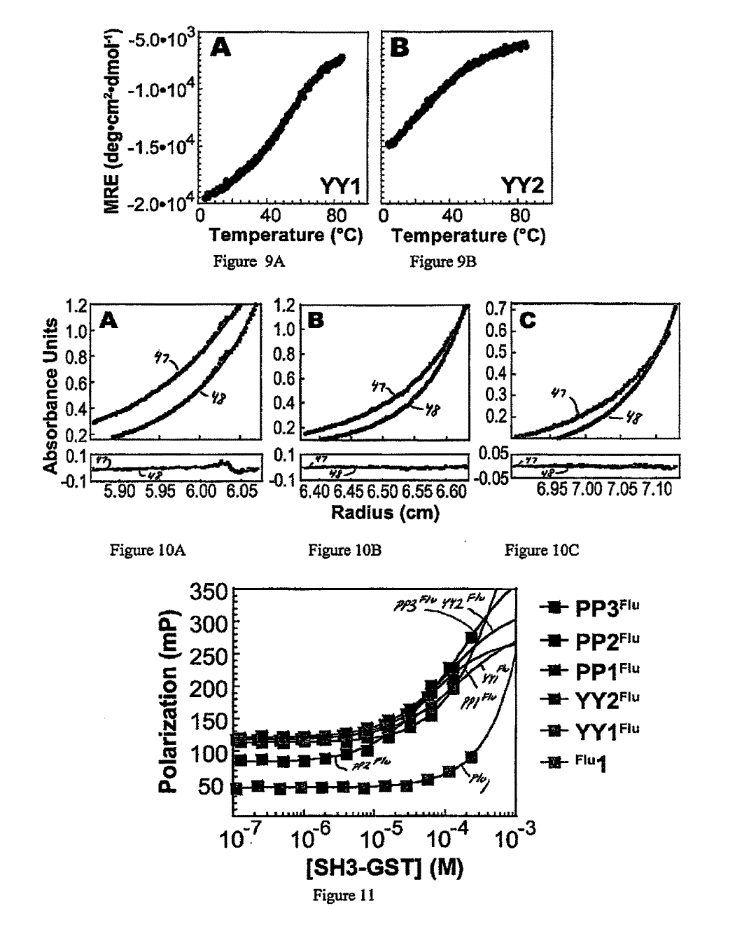

Circular dichroism spectra were measured using a Jasco J-810-150S Spectropolarimeter and Spectra Manager software v.1.53.01. Spectra were acquired at miniature protein concentrations of 5-20 micromolar in 10 mM sodium phosphate buffer (pH 7.4). Prior to analysis samples were spun 16,000×g for 10 minutes to ensure no particulates were transferred to the sample cuvette. Wavelength scans from 190 nm to 260 nm (data pitch 0.5 nm, scan speed 50 nm/min, 4 sec, 1 nm bandwidth and 3 accumulations) were taken every 10° C. as the sample temperature was raised from 4° C. to 84° C. using the variable temperature module. Simultaneously, the 0 readings were measured at 222 nm during the thermal denaturation (data pitch 0.5° C., 5 second delay, temperature slope 1° C./min, 4 sec response, 1 nm bandwidth, continuous scan mode). The sample was cooled back to 4° C. for another wavelength scan to verify that the melting transitions were reversible. Mean residue ellipticity values were calculated from the equation MRE=(Θₛₐₘₚₗₑ−Θ_{buffer})/(L×c×n×1000), where Θ is observed signal in millidegrees, L is the length of the cuvette, c is the concentration of peptide in dmol/cm³ and n is the number of amino acid residues in the molecule. The melting temperature (T_{M}) was estimated by the inflection point of the melt shown in FIG. 9, which shows temperature dependent circular dichroism spectra of miniature proteins (A) YY1 (25 micromolar) and (B) YY2 (20 micromolar) in 10 mM sodium phosphate buffer (pH 7.4). They were ˜50° C. and ˜25° C. for YY1 and YY2, respectively.

Analytical ultracentrifugation was performed using a Beckman Coulter Proteome Lab XL-I Protein Characterization System equipped with an AN 60-Ti 4-hole rotor and six-channel, carbon-epoxy composite centerpieces (Beckman). A stock of YY1 (1 mL, ˜500 micromolar) was dialyzed against 2×1 L dialysis buffer (1 mM KH₂PO₄, 155 mM NaCl, 3 mM Na₂HPO₄ (pH 7.4)) to remove residual TFA. The dialyzed peptide was separated into aliquots and diluted with dialysis buffer to prepare samples at 35.5 micromolar, 75 micromolar, and 142 micromolar concentrations, which were then centrifuged at 42,000 and 50,000 rpm. Temperature was maintained during centrifugation at 25° C. and the absorbance was read at 280 nm with 0.01 cm step size. The Match module of the Heteroanalysis software v1.1.19, which monitors radial concentration gradients, was used to determine when equilibrium had been established. The partial specific volume ( v̅) of the miniature proteins was calculated from amino acid composition using the Sedimentation Interpretation Program “Sednterp” software v1.08. The data was first fit to an equation describing the sedimentation of an ideal species of molecular weight Mₙ (Eq. 3) using Heteroanalysis software v1.1.19:

$$C ( r ) = C ( {r}^{0} ) exp \frac{( 1 - \overline{v} ρ ) {M}_{n} {ϖ}^{2}}{2 RT} ( {r}^{2} - {r}_{0}^{2} ) . \qquad Equation 3$$

In this equation, C represents the concentration (any unit) of the sedimenting species at radial positions r and r₀ cm from the center of rotation; v̅ is the partial specific volume of the sedimenting species in cc/gm; ρ is the density of the supporting buffer in g/cc; ω is the angular velocity of the rotor (radians/s); Mₙ, is the “molar” molecular weight of sedimenting species (g/mol); M_{b} is the “buoyant” molecular weight, equal to Mₙ(1− v̅ [symbol] ; R is the universal gas constant (8.315×10⁷ ergs mol⁻¹) and T is the temperature in Kelvin.

The best fit of the AU data for YY1 to Eq. 3 indicated a molecular weight of 5,968 Da, significantly higher than the calculated monomer molecular weight (4,327 Da), suggesting self-association of YY1 at these concentrations. The data was next fit to an equation describing sedimentation of a species in a monomer-n-mer equilibrium (Eq. 4). The best fit of the AU data to this equation yielded a stoichiometry of n=1.985 [1.758, 2.226] (two standard deviations calculated using f-statistics) with an lnK_{A}=8.624 [8.496, 8.753]. This value corresponds to a equilibrium dissociation constant (K_{D}) of 180 micromolar. Thus YY1 is approximately 80% monomeric at 25 micromolar concentration in PBS (pH 7.4).

$${C ( r )}_{tot} = {C ( {r}^{0} )}_{monomer} exp \frac{( 1 - \overline{v} ρ ) {M}_{n} {ϖ}^{2}}{2 RT} ( {r}^{2} - {r}_{0}^{2} ) + {C ( {r}^{0} )}_{n \text{-} mer} exp \frac{( 1 - \overline{v} ρ ) {nM}_{n} {ϖ}^{2}}{2 RT} ( {r}^{2} - {r}_{0}^{2} ) . \qquad Equation 4$$

FIG. 10 shows sedimentation equilibrium analysis of YY1 at (A) 141 micromolar; (B) 72 micromolar; and (C) 35.5 micromolar concentration, each at 42,000 (47) and 50,000 (48) rpm.

Fluorescence polarization assays were performed with 50 nM fluorescently labeled miniature protein and the indicated SH3 domain-GST fusion protein in PBS (1 mM KH₂PO₄, 155 mM NaCl, 3 mM Na₂HPO₄ (pH 7.4)) supplemented with 1 mM DTT and using the Analyst™ AD 96·384 automated fluorescence plate reader (LjL Biosystems). Binding reactions (32 microliters) were analyzed within 384-well assay plates (Corning Inc., Plate #3654) using settings defined by Criterion Host software v.2.00.11 and z height 1 mm. Polarization was measured by excitation with vertically polarized light at 485 nm and the fluorescence emission detected at 530 nm in both the vertical and horizontal directions. An average of five measurements were recorded for each well. Samples were equilibrated at room temperature for 25 min before analysis, a time sufficient for the binding reactions to reach equilibrium. The polarization data were fit using Kaleidagraph v3.6 software to Eq. 5, which is derived from first principles without assumptions.

F_{obs}=Pₘᵢₙ((Pₘₐₓ−Pₘᵢₙ)/(2[peptide^{Flu}])))([peptide^{Flu}]+[target protein]+K_{d}−(([peptide^{Flu}]+[target protein]+K_{d})²−4[peptide^{Flu}][target protein])^{0.5}) Equation 5.

In this equation, P_{obs} is the observed polarization value at any given concentration of target protein (GST•SH3 domain). Pₘᵢₙ is the polarization of peptide^{Flu} at 50 nM in the absence of target protein. Pₘₐₓ is the maximum polarization of peptide^{Flu} at saturation with target protein. K_{d} is the equilibrium dissociation constant. In the case of several miniature protein•SH3 domain pairs the binding curve did not reach saturation. In these cases a lower limit K_{d} was estimated by setting Pₘₐₓ equal to the observed polarization at the highest target protein concentration tested, which was often limited by solubility. The observed binding free energy, ΔG, was calculated from the relationship ΔG=−RT ln K_{d}⁻¹, where R is the universal gas constant (1.987×10⁻³ kcal mol⁻¹ K⁻¹) and T is the temperature in Kelvin (298 K). Fluorescence polarization analysis of the binding of fluorescently labeled miniature proteins and Peptide 1 to c-Src SH3^{P133L} are shown in FIG. 11. This figure shows the fluorescence polarization analysis of the binding of c-Src SH3^{P133L} to fluorescently labeled miniature proteins or peptide 1 (50 nM). Free fits are shown. K_{d} values were estimated as described.

Control experiments indicated that none of the fluorescently labeled miniature proteins or peptides used in this work bound detectably to GST (all K_{d}'s>100 micromolar). In several cases, the plots of polarization versus [SH3 domain] began to increase at high [SH3 domain] (>50 micromolar), perhaps because of a second binding event. In these cases (indicated by an asterisk (*) in Table 5), data above 50 micromolar target protein was excluded during the curve fits.

TABLE 5

| | | | | | | c-Src |
|---|---|---|---|---|---|---|
| | c-Src | Fyn | Hck | Lyn | Lck | P133L |
| ^{Flu}1 | 1.5 ± 0.1 | 1.4 ± 0.2 | 1.6 ± 0.2 | 2.0 ± 0.3 | >20 | >100 |
| PP1^{Flu} | 3.68 ± 0.06 | 25 ± 2 | 3.8 ± 0.2 | 4.9 ± 0.9 | 40 ± 3 | >55 |
| PP2^{Flu} | 1.3 ± 0.2 | 14 ± 3 | 1.3 ± 0.2 | 3.7 ± 0.9 | 2.7 ± 0.3 | >24 |
| PP3^{Flu} | 0.35 ± 0.04* | 2.1 ± 0.3 | 0.88 ± 0.06* | 1.8 ± 0.3* | 1.43 ± 0.08 | >60 |
| YY1^{Flu} | 6.8 ± 0.3 | 15 ± 2 | 1.6 ± 0.1 | 1.58 ± 0.02 | 23 ± 1 | >45 |
| YY2^{Flu} | 1.12 ± 0.05 | 7.1 ± 1.8 | 1.24 ± 0.05 | 1.55 ± 0.04 | 5.7 ± 1.4 | >45 |

Equilibrium dissociation constants (K_{d}, micromolar) of miniature protein•SH3 domain complexes

Activation of Hck kinase was measured using a validated NADH-coupled assay as described. Briefly, purified Hck kinase (15 micromolar) was first pre-incubated with 500 micromolar ATP and 20 mM MgCl₂ for about 30 min on ice to initiate autophosphorylation of Y416. The autophosphorylated product (0.55 micromolar final concentration) was then incubated in Hck Reaction Buffer (20 mM MgCl₂, 0.1 mM DTT, 20 μg/mL BSA, 100 mM HEPES (pH 7.5)) for 10 min at room temperature with the indicated concentration of Nef or miniature protein. At this time the remaining assay components (1 mM PEP, 0.2 mM NADH, 6 U pyruvate kinase (Type II from rabbit muscle, Sigma), 6 U L-lactic dehydrogenase (Type II from rabbit muscle, Sigma), 600 micromolar substrate peptide, and 500 micromolar ATP) were added to initiate the reaction (total volume 200 microliters) and the absorbance at 340 nm was monitored every 10 sec for 10 min using a Beckman DU 730 UVN is Spectrophotomer. The activation constant K_{act} for each ligand was calculated from the initial rate as a function of ligand concentration as previously described, using Eq. 6. Fold activation was calculated by dividing the rate in the presence of ligand by the rate in the absence of ligand.

vₐ=V_{act}[L]/(K_{act}±[L]) Equation 6.

In this equation vₐ is velocity measured in the presence of ligand minus the velocity measured in its absence; V_{act} is the maximal activated velocity minus the velocity measured in the absence of ligand; [L] is the concentration of activator and K_{act} is concentration of activator at half-maximal activation.

Table 6 shows activation of Hck kinase by miniature proteins and peptide 1.

TABLE 6

| Activator (50 | | |
|---|---|---|
| micromolar) | Activity (nmol · sec⁻¹) | Fold-Activation |
| none | 10.9 ± 4.6 | 1 ± 6 |
| peptide 1 | 33.9 ± 3.2 | 3 ± 1 |
| PP1 | 83.6 ± 6.0 | 8 ± 3 |
| PP2 | 138.8 ± 7.6 | 13 ± 5 |
| PP3 | 83.9 ± 5.4 | 7 ± 3 |
| YY1 | 34.1 ± 1.7 | 3 ± 1 |
| YY2 | 114.7 ± 7.7 | 10 ± 4 |
| Nef | 231.7 ± 6.0 | 21 ± 1 |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
(SEQ ID NO: 18)
APPLPPRNRPGEDASPEELSRYYASLRHYLNLVTRQRY.

2. The composition of claim 1, further comprising a pharmaceutically acceptable carrier.

3. A composition, comprising:
a protein having a total length of no more than about 40 amino acids and a sequence:
X¹X²X³,
wherein:
X¹ comprises a sequence J¹PJ²J³PJ⁴J⁵J⁶ (SEQ ID NO: 20) or J¹ PJ²J³PJ⁴J⁵J⁶J⁷ (SEQ ID NO: 21)
X² is a hinge region comprising at least one proline;
at least about 50% of X³ comprises at least 10 residues of an alpha helix of PYY; and
each of J¹-J⁷ is independently an amino acid residue.

4. The composition of claim 3, wherein the protein has a total length of no more than about 40 residues.

5. The composition of claim 3, wherein X² consists of proline.

6. The composition of claim 3, wherein X² has a length of no more than 3 residues.

7. The composition of claim 3, wherein X¹ has a length of no more than 10 residues.

8. The composition of claim 3, wherein X¹ comprises a sequence PPJ²J³PJ⁴J⁵J⁶ (SEQ ID NO: 22) or PPJ²J³PJ⁴J⁵J⁶J⁷ (SEQ ID NO: 23)

9. The composition of claim 3, wherein X¹ comprises a sequence J¹PJ²PPJ⁴J⁵J⁶ (SEQ ID NO: 24) or J¹PJ²PPJ⁴J⁵J⁶J⁷ (SEQ ID NO: 25)

10. The composition of claim 3, wherein X¹ comprises a sequence PPJ²PPJ⁴J⁵J⁶ (SEQ ID NO: 26) or PPJ²PPJ⁴J⁵J⁶J⁷ (SEQ ID NO: 27).

11. The composition of claim 3, wherein X¹ comprises a sequence PPLPPRNR (SEQ ID NO: 28).

12. The composition of claim 3, wherein X³ comprises a sequence PEELSRYYASLRHYLN (SEQ ID NO: 29).

13. The composition of claim 3, wherein X³ comprises a sequence GEDASPEELSRYYASLRHYLNLVTRQRY (SEQ ID NO: 30).

14. The composition of claim 3, wherein X³ comprises a sequence SRYYASLRHYLNLVTRQRY (SEQ ID NO: 33).

15. The composition of claim 3, wherein X³ comprises a sequence GDDAPVEDLIRFYDNLQQYLNVVTRHRY (SEQ ID NO: 31).

16. The composition of claim 3, wherein X³ comprises a sequence IRFYDNLQQYLNVVTRHRY (SEQ ID NO: 34).

17. The composition of claim 3, wherein X³ comprises at least 4 arginine residues.

18. The composition of claim 17, wherein X³ has between 4 and 6 arginine residues.

19. The composition of claim 3, wherein X³ comprises a sequence GEDASPEELSRYYASLRHYLNLVTRQRY (SEQ ID NO: 30) modified such that at least 1 and no more than 6 residues of X³ have been substituted by arginine.

20-24. (canceled)

25. A composition, comprising:
an avian pancreatic polypeptide (aPP) modified by substitution of one to four amino acid residues, wherein the modified miniature protein, when in phosphate-buffered saline solution at a concentration of 10⁻⁶ M, does not substantially form a multimer.

26-45. (canceled)


SEQUENCE LISTING (34 sequences)

SEQ ID NO: 1 - protein, 36 aa, Unknown
  Avian Pancreatic Peptide
GPSQPTYPGDDAPVEDLIRFYDNLQQYLNVVTRHRY

SEQ ID NO: 2 - protein, 36 aa, Unknown
  PYY
YPAKPEAPGEDASPEELSRYYASLRHYLNLVTRQRY

SEQ ID NO: 3 - protein, 30 aa, Unknown
  pGolemi
PFPPTPPGEEAPVEDLIRFYNDLQQYLNVV

SEQ ID NO: 4 - protein, 37 aa, Unknown
  p007
GGSRATMPGDDAPVEDLKRFRNTLAARRSRARKAARA

SEQ ID NO: 5 - protein, 36 aa, Artificial Sequence
  aPPY7A
GPSQPTAPGDDAPVEDLIRFYDNLQQYLNVVTRHRY

SEQ ID NO: 6 - protein, 36 aa, Artificial Sequence
  aPPF20A
GPSQPTYPGDDAPVEDLIRAYDNLQQYLNVVTRHRY

SEQ ID NO: 7 - protein, 36 aa, Artificial Sequence
  aPPY21A
GPSQPTYPGDDAPVEDLIRFADNLQQYLNVVTRHRY

SEQ ID NO: 8 - protein, 36 aa, Artificial Sequence
  aPPY7AT6E
CPSQPEAPGDDAPVEDLIRFYDNLQQYLNVVTRHRY

SEQ ID NO: 9 - protein, 36 aa, Artificial Sequence
  aPPY7AQ25R
GPSQPTAPGDDAPVEDLIRFYDNLRQYLNVVTRHRY

SEQ ID NO: 10 - protein, 36 aa, Artificial Sequence
  aPPY7AP13SV14P
GPSQPTAPGDDASPEDLIRFYDNLQQYLNVVTRHRY

SEQ ID NO: 11 - protein, 36 aa, Artificial Sequence
  aPPY7AP13VV14P
GPSQPTAPGDDAVPEDLIRFYDNLQQYLNVVTRHRY

SEQ ID NO: 12 - protein, 37 aa, Artificial Sequence
  PP1
APPLPPRNPGDDAPVEDLIRFYDNLQQYLNVVTRHRY

SEQ ID NO: 13 - protein, 37 aa, Artificial Sequence
  PP2
APPLPPRNRGDDAPVEDLIRFYDNLQQYLNVVTRHRY

SEQ ID NO: 14 - protein, 37 aa, Artificial Sequence
  PP3
APPLPPRNRPRDAPVEDLIRFYDNLQQYLNVVTRHRY

SEQ ID NO: 15 - protein, 37 aa, Artificial Sequence
  YY1
APPLPPRNPGEDASPEELSRYYASLRHYLNLVTRQRY

SEQ ID NO: 16 - protein, 37 aa, Artificial Sequence
  YY2
APPLPPRNRGEDASPEELSRYYASLRHYLNLVTRQRY

SEQ ID NO: 17 - protein, 12 aa, Artificial Sequence
  Peptide 1
APPLPPRNRPRL

SEQ ID NO: 18 - protein, 38 aa, Artificial Sequence
  YY3
APPLPPRNRPGEDASPEELSRYYASLRHYLNLVTRQRY

SEQ ID NO: 19 - protein, 16 aa, Unknown
  Hck Substrate Peptide
AEEEIYGEFEAKKKKG

SEQ ID NO: 20 - protein, 8 aa, Artificial Sequence
  Synthetic Peptide
XPXXPXXX

SEQ ID NO: 21 - protein, 9 aa, Artificial Sequence
  Synthetic Peptide
XPXXPXXXX

SEQ ID NO: 22 - protein, 8 aa, Artificial Sequence
  Synthetic Peptide
PPXXPXXX

SEQ ID NO: 23 - protein, 9 aa, Artificial Sequence
  Synthetic Peptide
PPXXPXXXX

SEQ ID NO: 24 - protein, 8 aa, Artificial Sequence
  Synthetic Peptide
XPXPPXXX

SEQ ID NO: 25 - protein, 9 aa, Artificial Sequence
  Synthetic Peptide
XPXPPXXXX

SEQ ID NO: 26 - protein, 8 aa, Artificial Sequence
  Synthetic Peptide
PPXPPXXX

SEQ ID NO: 27 - protein, 9 aa, Artificial Sequence
  Synthetic Peptide
PPXPPXXXX

SEQ ID NO: 28 - protein, 8 aa, Artificial Sequence
  Polyproline Helix Fragment
PPLPPRNR

SEQ ID NO: 29 - protein, 16 aa, Artificial Sequence
  PYY Fragment
PEELSRYYASLRHYLN

SEQ ID NO: 30 - protein, 28 aa, Artificial Sequence
  PYY Fragment
GEDASPEELSRYYASLRHYLNLVTRQRY

SEQ ID NO: 31 - protein, 28 aa, Artificial Sequence
  aPP Fragment
GDDAPVEDLIRFYDNLQQYLNVVTRHRY

SEQ ID NO: 32 - protein, 36 aa, Artificial Sequence
  Synthetic Peptide
GPSQPTXPGDDAXPEDLIRFYDNLQQYLNVVTRHRY

SEQ ID NO: 33 - protein, 19 aa, Artificial Sequence
  Synthetic Peptide
SRYYASLRHYLNLVTRQRY

SEQ ID NO: 34 - protein, 19 aa, Artificial Sequence
  Synthetic Peptide
IRFYDNLQQYLNVVTRHRY